SEQUENCE LISTING (11 sequences)

SEQ ID NO: 1 - DNA, 825 bp, Ancylostoma caninum
  CDS (1) . . (822)
atggaggcctatcttgtggtcttaattgccattgctggcatagctcattccaatgaacacaacctgaggtgcccgcagaatggaacagaaatgcccggtttcaacgactcgattaggcttcaatttttagcaatgcacaatggttacagatcaaaacttgcgctaggtcacatcagcataactgaagaatccgaaagtgacgatgatgacgatttcggttttttacccgatttcgctccaagggcatcgaaaatgagatatctggaatatgactgtgaagctgaaaaaagcgcctacatgtcggctagaaattgctcggacagttcttctccaccagagggctacgatgaaaacaagtatattttcgaaaactcaaacaatatcagtgaagctgctctgaaggccatgatctcgtgggcaaaagaggctttcaacctaaataaaacaaaagaaggagaaggagttctgtaccggtcgaaccacgacatatcaaacttcgctaatctggcttgggacgcgcgtgaaaagtttggttgcgcagttgttaactgccctttgggagaaatcgatgatgaaaccaaccatgatggagaaacctatgcaacaaccatccatgtagtctgccactacccgaaaataaacaaaactgaaggacagccgatttacaaggtagggacaccatgcgacgattgcagtgaatacacaaaaaaagcagacaataccacgtctgcggatccggtgtgtattccggatgacggagtctgctttattggctcgaaagccgattacgatagcaaggagttttatcgattccgagagttatga

SEQ ID NO: 2 - protein, 274 aa, Ancy1ostoma caninum
MEAYLVVLIAIAGIAHSNEHNLRCPQNGTEMPGFNDSIRLQFLAMHNGYRSKLALGHISITEESESDDDDDFGFLPDFAPRASKMRYLEYDCEAEKSAYMSARNCSDSSSPPEGYDENKYIFENSNNISEAALKAMISWAKEAFNLNKTKEGEGVLYRSNHDISNFANLAWDAREKFGCAVVNCPLGEIDDETNHDGETYATTIHVVCHYPKINKTEGQPIYKVGTPCDDCSEYTKKADNTTSADPVCIPDDGVCFIGSKADYDSKEFYRFREL

SEQ ID NO: 3 - protein, 257 aa, Ancylostoma caninum
  PEPTIDE (1)..(257)
NEHNLRCPQNGTEMPGFNDSIRLEFLAMHNGYRSKLALGHISITEESESDDDDDFGFLPDFAPRASKMRYLEYDCEAEKSAYMSARNCSDSSSPPEGYDENKYIFENSNNISEAALKAMISWAKEAFNLNKTKEGEGVLYRSNHDISNFANLAWDAREKFGCAVVNCPLGEIDDETNHDGETYATTIHVVCHYPKINKTEGQPIYKVGTPCDDCSEYTKKADNTTSADPVCIPDDGVCFIGSKADYDSKEFYRFREL

SEQ ID NO: 4 - protein, 8 aa, Ancylostoma caninum
KAMISWAK

SEQ ID NO: 5 - protein, 8 aa, Ancylostoma caninum
EFYRFREL

SEQ ID NO: 6 - protein, 11 aa, Ancylostoma caninum
DISNFANLAWD

SEQ ID NO: 7 - protein, 30 aa, Ancylostoma caninum
DENKYIFENSNNISEAALKAMISWAKEAFN

SEQ ID NO: 8 - DNA, 885 bp, Partially Ancylostoma caninum
ggcgaattcaccatggaggcctatcttgtggtcttaattgccattgctggcatagctcattccaatgaacacaacctgaggtgcccgcagaatggaacagaaatgcccggtttcaacgactcgattaggcttcaatttttagcaatgcacaatggttacagatcaaaacttgcgctaggtcacatcagcataactgaagaatccgaaagtgacgatgatgacgatttcggttttttacccgatttcgctccaagggcatcgaaaatgagatatctggaatatgactgtgaagctgaaaaaagcgcctacatgtcggctagaaattgctcggacagttcttctccaccagagggctacgatgaaaacaagtatattttcgaaaactcaaacaatatcagtgaagctgctctgaaggccatgatctcgtgggcaaaagaggctttcaacctaaataaaacaaaagaaggagaaggagttctgtaccggtcgaaccacgacatatcaaacttcgctaatctggcttgggacgcgcgtgaaaagtttggttgcgcagttgttaactgccctttgggagaaatcgatgatgaaaccaaccatgatggagaaacctatgcaacaaccatccatgtagtctgccactacccgaaaataaacaaaactgaaggacagccgatttacaaggtagggacaccatgcgacgattgcagtgaatacacaaaaaaagcagacaataccacgtctgcggatccggtgtgtattccggatgacggagtctgctttattggctcgaaagccgattacgatagcaaggagttttatcgattccgagagttatgaataagtcgagacgtataaagaagccaaggcaacgtaagcgagaatttc

SEQ ID NO: 9 - DNA, 1845 bp, Ancylostoma caninum
agttctcagatagtcacagtagcccttcttttcattgtacacaagtgaagatgggcacttcatggtagtcgcgactccttcattacagtaaacatagtcggatgtgcatcccaacgaatagtagccattctgctttgtcttgcagtcaacggtcttcgcaatttgtggtacagcagcaggagccggaggctgcatcgctggagctgctggtggagctggcacaacagaagccggaggtggagcaaccagttcaggcgtgcagttctcaggatagtcgcagtagcccttcttctcatggtatacaagtgaagaatggaggcctatcttgtggtcttaattgccattgctggcatagctcattccaatgaacacaacctgaggtgcccgcagaatggaacagaaatgcccggtttcaacgactcgattaggcttcaatttttagcaatgcacaatggttacagatcaaaacttgcgctaggtcacatcagcataactgaagaatccgaaagtgacgatgatgacgatttcggttttttacccgatttcgctccaagggcatcgaaaatgagatatctggaatatgactgtgaagctgaaaaaagcgcctacatgtcggctagaaattgctcggacagttcttctccaccagagggctacgatgaaaacaagtatattttcgaaaactcaaacaatatcagtgaagctgctctgaaggccatgatctcgtgggcaaaagaggctttcaacctaaataaaacaaaagaaggagaaggagttctgtaccggtcgaaccacgacatatcaaacttcgctaatctggcttgggacgcgcgtgaaaagtttggttgtcgcagttgttaactgccctttgggagaaatcgatgatgaaaccaaccatgatggagaaacctatgcaacaaccatccatgtagtctgccactacccgaaaataaacaaaactgaaggacagccgatttacaaggtagggacaccatgcgacgattgcagtgatacacaaaaaaagcagacaataccacgtctgcggatccggtgtgtattccggatgacggagtctgctttattggctcgaaagccgattacgatagcaaggagttttatcgattccgagagttatgaataagtcgagacgtataaagaagccaaggcaacgtaagcgagcaagtctcgaagacgatggagtcagcgaaagaggcggctgccaaagttggcgagcaggtgtcagattttttccaagggaacccattttccacgcctgtgggccgcaagatagaacttgccacgaacgcttcgattcttgcactgagaattggggtttgaacatggaaatctgtgatttcgtcaataacactgaggacggtgccaaagatgctgtacgggctattcgcaaacgtctgcacacaaatatgtgtaagaataacgcaatcgtcatgtacacattaacggtgctggagacgtgcgtgaagaactgtggccataatttccacgtgctcgtatgttccaaggactttgtgcaggatttggtgaagttgatcggctcgaagttcgatacgcctcagattattcacgagcgtgtattgtcacttattcaggcttgggcagatgcattccgcaatcaaccagatcttcagggagtcgtacaggtctatgaagaacttgttagtaagggggttacattccctgcaactgatctagacgctatggcacctatactaacaccaaaacaaacagtcttcactgagccaaaggcatcaacggctgttccttcgcagtcaggtggaggacctagttacgaggtggtcagccaaccagatggtccaatttact

SEQ ID NO: 10 - DNA, 43 bp, Artificial Sequence
  Description of Artificial Sequence Synthetic sequences from cloning vectors
ctgcagtcaccgtccttgacacaagcttgatatcgaattcacc

SEQ ID NO: 11 - DNA, 59 bp, Artificial Sequence
  Description of Artificial Sequence Synthetic sequences from cloning vectors
ataagtcgagacgtataaagaagccaaggcaacgtaagcgagaattcctgcagcccggg

(19) United States
(12) Patent Application Publication
(10) Pub. No.: US 2004/0086964 A1
(43) Pub. Date: May 6, 2004

(54) Process for the preparation of neutrophil inhibitory factor

(76) Inventors: Stefanie Beate Pluschkell, Groton, CT (US); Roderick William Geldart, West Kingston, RI (US); Lewis Ho, Waterford, CT (US); Mark Alan Koehler, Gaithersburg, MD (US); Centenary Afam Okediadi, Franklin, CT (US); Stephen Joseph Pias, Gales Ferry, CT (US); Marie Meiying Zhu, East Lyme, CT (US); Steven Joseph Hawrylik, N.Stonington, CT (US); Matthew Moyle, Houston, MA (US)

Correspondence Address: Suzanne L Biggs, Pillsbury Winthrop, Suite 200, 11682 EL Camino Real, San Diego, CA, 92130, US

(21) Appl. No.: 10/362,263

(22) PCT Filed: Aug. 15, 2001
(86) PCT No.: PCT/US01/25733

(30) Foreign Application Priority Data
Aug. 23, 2000 (US) 09/644,942
Feb. 28, 2001 (US) 09/797,410

(51) Int. Cl. C12P 21/02; C12N 5/06; C07K 14/475

(52) U.S. Cl. 435/69.1; 435/320.1; 435/325; 530/397

(57) ABSTRACT

The present invention relates to a method for the preparation of a Neutrophil Inhibitory Factor (NIF) comprising the cultivation of mammalian cells expressing NIF in an animal component-free growth medium. The present invention may be employed in large-scale preparation of NIF. The invention also relates to a method for the preparation of recombinant proteins comprising the cultivation of mammalian cells expressing an exogenous recombinant protein in an animal component-free growth medium.

### FIELD OF THE INVENTION

The Application is a continuation-in-part of U.S. Ser. No. 09/644,942, filed Aug. 23, 2000. The disclosure of which is incorporated herein by reference.

The present invention relates to a method for the preparation of a Neutrophil Inhibitory Factor (NIF) comprising the cultivation of mammalian cells in an animal component-free growth medium. The present invention may be employed in large-scale preparation of NIF. In addition, the present invention provides a general method for the preparation of recombinant proteins comprising the cultivation in an animal component-free medium of mammalian cells, in particular CHO cells, expressing an exogenous recombinant protein.

### BACKGROUND AND INTRODUCTION TO THE INVENTION

NIFs are proteins that are specific inhibitors of the activity of neutrophil cells. Neutrophils are a member of the group of cell types known as granulocytes, a subclass of the leukocyte family of cells.

Neutrophils are an important component of the defense system in a host against microbial attack. In response to soluble inflammatory mediators released at the site of injury by cells, neutrophils enter into the area of the injured tissue from the bloodstream and when activated, kill foreign cells by phagocytosis and/or the release of cytotoxic compounds, such as oxidants, proteases and cytokines. Although the activity of neutrophils is important to fight infection, they also are known to damage the host tissue. Neutrophils may give rise to an abnormal inflammatory response whereby significant tissue damage may be caused by the release of toxic substances at the vascular wall or in uninjured tissue. Alternatively, neutrophils which adhere to a capillary wall or aggregate in venules can produce ischemic tissue damage.

Abnormal inflammatory response is implicated in the pathogenesis of a variety of clinical disorders including adult respiratory distress syndrome (ARDS); ischemia-reperfusion injury following myocardial infarction, shock, stroke, and organ transplantation; acute and chronic allograft rejection; vasculitis; sepsis; rheumatoid arthritis; head trauma; and inflammatory skin diseases. Harlan et al., Immunol. Rev., 114:5 (1990).

One of the specific activities that NIFs have been reported to inhibit is adhesion of neutrophils to vascular endothelial cells.

Certain NIFs have been isolated from hookworms and related species, in particular the canine hookworm (Ancylostoma caninum), Moyle et al., J. Biol. Chem., 269:10008-15 (1994), and have been made by recombinant methods. When isolated from parasitic worms, the NIF is a glycoprotein. Recombinant NIFs produced by certain expression systems have been reported to exhibit post-translational glycosylation and sialylation.

NIFs have been reported to inhibit other aspects of neutrophil activity, including the release of hydrogen peroxide, release of superoxide anion, release of myeloperoxidase, release of elastase, homotypic neutrophil aggregation, adhesion to plastic surfaces, adhesion to vascular endothelial cells, chemotaxis, transmigration across a monolayer of endothelial cells and phagocytosis. In particular NIF has been shown to be effective in reducing infarct size in a rat reperfusion model of stroke. Jiang et al., Ann. Neurology, 38:935-942 (1995); Jiang et al., Brain Res., 788:25-34 (1998).

Certain Neutrophil Inhibitory Factors are described in greater detail, along with methods of isolating them from natural sources and of cloning them by recombinant methods, in U.S. Pat. No. 5,919,900, issued Jul. 6, 1999, U.S. Pat. No. 5,747,296, issued May 5, 1998, and U.S. Pat. No. 5,789,178, issued Aug. 4, 1998. These patent documents are incorporated herein by reference in their entirety.

Heretofore, the cultivation of cells expressing NIF has been carried out in growth media containing bovine serum albumin. See, e.g., U.S. Pat. No. 5,919,900.

As a general matter, the cultivation of cells expressing recombinant proteins is most often conducted in media which contain either animal-derived serum or animal-extracted proteins. However, in view of the increasing concerns in general over the use of animal components and in particular over the contamination of bovine products by pathogens, including contamination by the organism giving rise to outbreaks of bovine spongiform encephalopathy (BSE), there is a need for growth medium which is free of animal components, e.g., serums and proteins. In U.S. Pat. No. 5,122,469, serum-free media are recited. However, serum-free media have often not been optimized for cell growth, protein production, and post-translational modification.

The present invention provides a animal serum-free and animal protein-free medium as well as a method of preparation of NIF using said serum- and protein-free medium to provide NIF in high yields.

### SUMMARY OF THE INVENTION

The present invention is directed to a process for the preparation of Neutrophil Inhibitory Factor (NIF) comprising the step of incubating a cell line expressing NIF in an animal component-free growth medium. Preferably, the NIF produced via the present invention is a 257-amino acid protein, mature NIF-1FL (also termed “NIF1”) (SEQ. ID. NO. 3). The NIF so produced is glycosylated and has a relative molecular weight of about 38.3 to about 64.1 kDa. The glycan structures are typically branched and may be capped by sialic acid residues. The degree of glycosylation.may vary, but preferably, the NIF produced has a distribution of mono, di, tri, and tetra-antennary glycan structures. More preferably, the NIF produced is about 5 to about 25% mono-sialylated, about 10 to about 30% di-sialylated, about 15 to about 35% tri-sialylated, about 15 to about 45% tetra-sialylated and about 1 to about 20% non-sialylated.

According to a preferred aspect of the invention, the cell line expressing NIF is a Chinese Hamster Ovary (“CHO”)cell line comprising the NIF gene, more preferably a cell line which is not anchorage-dependent.

According to a most preferred aspect of the present invention, the cell line is the CHO-K1 cell line (ATCC CCL-61) modified by transfection with the glutamine synthetase/methionine sulfoximine co-amplification vector pEE14 expressing the NIF1 gene. WO 87/04462 and 89/10404 describe recombinant DNA sequences, vectors and use of the glutamine synthetase system in expression systems.

The most preferred cell line for use according to the processes and methods of the present invention is the cell line PFG01 (ATCC PTA-2503).

The preparation and cultivation of the most preferred cell line expressing NIF is described in the Examples 1 and 2 below.

A preferred embodiment of the invention is wherein the animal component-free production growth medium comprises:

(i) a CHO-III-PFM/glucose solution;

(ii) sodium hypoxanthine;

(iii) thymidine; and

(iv) yeast extract.

“CHO-III-PFM” refers to a protein-free medium optimized for suspension culture of CHO cells which is made without hypoxanthine and thymidine and which is available from Life Technologies (Grand Island, N.Y.). “CHO-III-PFM glucose solution” refers to a CHO-III-PFM medium made with added glucose, a preparation also available from Life Technologies. A preferred CHO-III-PFM/glucose solution is custom formula No. 98-0289 (Life Technologies, Rockville, Md., Grand Island, N.Y., a division of Irivitrogen Corp., Carlsbad, Calif.) which is a CHO-III-PFM/glucose solution having additional glucose (3.45 g/L D-glucose) and which does not contain hypoxanthine, thymidine or L-glutamine.

The CHO-III-PFM/glucose solution is itself animal component-free (free of animal serum and animal protein). It should be noted, however, that other commercially available CHO cell cultivation media which are animal component-free and which incorporate the above-noted attributes and components of the CHO-III-PFM media may also be used within the scope of the invention.

A preferred yeast extract is that purchased under the trade name Bacto (Difco/Becton-Dickinson). Other commercially available yeast extracts also may be used.

A solution of phenol red, preferably a solution of about 0.5% w/v thereof, may be added to the media for use as a visual pH indicator. Such a phenol red solution is more preferably used in the amount of about 0 to about 3.0 ml per liter of media.

A more preferred embodiment of the invention is wherein the animal component-free production growth medium comprises:

(i) CHO-III-PFM/glucose solution;

(ii) about 50 to about 100 μmol sodium hypoxanthine per liter (i);

(iii) about 8 to about 32 μmol thymidine per liter (i); and

(iv) about 0.5 to about 5.0 grams per liter (i) yeast extract.

According to a preferred aspect, sodium hypoxanthine and thymidine are added as a 10 mM sodium hypoxanthine/1.6 mM thymidine solution. Preferably about 5 to about 20 ml of the sodium hypoxanthine/thymidine solution per liter (i) are added.

A most preferred embodiment of the invention is wherein the animal component-free-production growth medium comprises:

(i) CHO-III-PFM/glucose solution;

(ii) about 10.0 ml per liter (i) of a 10 mM sodium hypoxanthine/1.6 mM thymidine solution; and

(iii) about 1.5 grams per liter (i) yeast extract.

Optionally, about 0.5 ml per liter (i) of a 0.5% w/v solution of phenol red may be added to the medium.

The present invention is also directed to a process for the preparation of Neutrophil Inhibitory Factor (NIF) comprising the steps of:

(i) providing an inoculum prepared by incubating a cell line expressing NIF in an animal component-free inoculum growth medium; and

(ii) transferring said inoculum to a vessel containing an animal component-free production growth medium.

According to a preferred embodiment of this aspect of the invention is the inoculum growth medium comprises:

(i) a CHO-III-PFM/glucose solution;

(ii) sodium hypoxanthine;

(iii) thymidine;

(iv) an amino acid solution comprising acids selected from the group consisting of L-aspartic acid, L-glutamic acid, L-asparagine, L-proline, L-serine, and L-methionine;

(v) optionally, L-methionine sulphoximine (“MSX”); and

(vi) L-cysteine.

Optionally, a solution containing phenol red, preferably a solution of about 0.5% w/v thereof, may be. added to this inoculum medium for use as a visual pH indicator; preferably the solution is added in the amount of about 0 to about 3.0 ml per liter of the medium.

A more preferred embodiment of this aspect of the invention is wherein the inoculum growth medium comprises:

(i) CHO-III-PFM/glucose solution;

(ii) about 50 to about 100 μmol sodium hypoxanthine per liter (i);

(iii) about 8 to about 32 μmol thymidine per liter (i);

(iv) addition of the following amino acids in the noted amounts per liter (i): L-aspartic acid (about 15 to about 90 mg); L-glutamic acid (about 12 to about 75 mg), L-asparagine (about 50 to about 300 mg), L-proline (about 6 to about 38 mg), L-serine (about 15 to about 90 mg) and L-methionine (about 7 to about 45 mg);

(v) about 0 to about 75 μmol L-methionine sulphoximine (MSX) per liter (i); and

(vi) about 10 to about 40 mg cysteine per liter (i).

The amino acids of (iv) may be conveniently added as about 5 to about 30 ml per liter (i) of an amino acid solution comprising L-aspartic acid (about 3.0 g/l), L-glutamic acid (about 2.50 g/l), L-asparagine (about 10.00 g/l), L-proline (about 1.25 g/l), L-serine (about 3.0 g/l), and L-methionine (about 1.50 g/l). MSX may be optionally added as about 0.5 to about 3 ml per liter (i) of a 25 mM L-methionine sulphoximine (MSX) solution to give about 12.5 to about 75 μmole MSX per liter. Sodium hypoxanthine and thymidine may be conveniently added as about 5 to about 20 ml of a 10 mM sodium hypoxanthine/1. 6 mM thymidine solution.

A most preferred embodiment of the invention is wherein the inoculum growth medium comprises:

(i) CHO-III-PFM/glucose solution;

(ii) about 10.0 ml per liter (i) of a 10 mM sodium hypoxanthine/1.6 mM thymidine solution;

(iii) about 20.0 ml per liter (i) of an amino acid solution comprising L-aspartic acid (about 3.0 g/l), L-glutamic acid (about 2.5 g/l), L-asparagine (about 10.0 g/l), L-proline (about 1.25 g/l), L-serine (about 3.0 g/l), and L-methionine (about 1.5 g/l);

(iv) optionally about 1.0 ml per liter (i) of an 25 mM L-methionine sulphoximine (MSX) solution; and

(v) about 25.0 mg per liter (i) of L-cysteine. Optionally, about 0.5 ml per liter (i) of a solution of about 0.5% w/v phenol red may be added to the inoculum medium.

The present invention further relates to an animal component-free growth medium, as described above. In addition, the present invention relates to an animal component-free inoculum growth medium.

Further, the present invention also relates to a method for the preparation of a recombinant protein comprising the cultivation of mammalian cells expressing an exogenous recombinant protein in an animal component-free growth medium of the present invention. In a preferred embodiment, the mammalian cells are Chinese Hamster Ovary cells transfected with a glutamine synthetase plasmid vector comprising a nucleic molecule having the DNA coding region for the recombinant protein. Preferred vectors are a glutamine synthetase/methionine sulfoximine co-amplification vector, such as pEE14 or pEE14.1 (Lonza Biologics, Slough, UK).

Definitions

“Neutrophil Inhibitory Factor” or “NIF” refers to a protein which may be isolated from natural sources or made by recombinant methods. Neutrophil Inhibitory Factor is a protein which is neither an antibody, a member of the integrin or selectin families, nor a member of the immunoglobulin superfamily of adhesive proteins and which, when isolated from a parasitic worm, is glycosylated. Recombinant NIF may or may not be glycosylated or may be glycosylated to a variable degree; this may be affected by the expression system and/or culture conditions used in producing recombinant NIF.

NIF1 or mature NIF-1FL refers to a protein which is expressed in a proform, NIF-1FL (SEQ. ID. NO. 2), and then, after synthesis, is cleaved (while within the cell) to give mature NIF-1FL or NIF1 (SEQ. ID. NO. 3).

“NIF1cr” refers to the coding sequence for NIF1.

The term “NIF gene” refers to a nucleic acid molecule which encodes a Neutrophil Inhibitory Factor. Certain nucleic acid molecules which encode a NIF are described in U.S. Pat. No. 5,919,900.

The term “cell line expressing NIF” refers to a cell line which has been transformed with a nucleic acid molecule encoding a NIF so as to express a Neutrophil Inhibitory Factor.

The cell line PFG01 is a CHO-K1 (ATCC-CCL-61) cell line which has been transfected with the glutamine synthetase/methionine sulfoximine co-amplication vector pEE14 expressing the NIF1 gene. Pfizer Inc. a Delaware corporation, doing business at 235 East 42^{nd} Street, New York, N.Y. made a deposit with the American Type Culture Collection of cell line PFG01 (ATCC PTA-2503) on Sep. 27, 2000.

The term “CHO-III-PFM/glucose solution” refers to a growth medium manufactured by Life Technologies (Grand Island, N.Y.; PFM=protein-free medium) with added glucose developed specifically for the cultivation of CHO cells.

The term “yeast extract” refers to a complex supplement containing peptides which is extracted from yeast cells and is free of animal-derived compounds.

### BRIEF DESCRIPTION OF THE DRAWINGS

FIG. 1 depicts the coding sequence for NIF1 (SEQ. ID. NO. 1), the corresponding amino acid translation (SEQ. ID. NO. 2) and the amino acid sequence of mature NIF1 (SEQ. ID. NO. 3). Nucleotides are numbered from the 5′-end, and amino acids are numbered from the start of the mature polypeptide (SEQ. ID. NO. 3.) (The N-terminal Asn is indicated.) Numbers along the left-hand margin denote the nucleotide number of the nucleic acid sequence or amino acid number. (bold) of the mature NIF1 sequence of the first entry on each line. Peptides identified by amino acid sequencing are underlined. The peptides T-20 (SEQ. ID. NO. 4), T-22 (SEQ. ID. NO. 5), D-96 (SEQ. ID. NO. 6), and D-102 (SEQ. ID. NO. 7) were used to design forward and reverse primers for initial and subsequent cloning purposes. Nucleotide sequences in lower case represent the nucleotides added by the PCR primers during rescue of the coding region for cloning into the BSII shuttle vector (SEQ. ID. NO. 8). This figure represents the sequence determined from both strands of DNA using a {BSII/}{pEE14/}{pSG5}NIF1cr construct.

FIG. 2 depicts the sequence for the full length cDNA (SEQ. ID. NO. 9), as obtained from Ancylostoma mRNA preparations, after cloning of the cDNA into λgt10/EcoRI vectors, and subcloning into the BSII rescue vector. The nucleotide sequence of NIF1 was determined-using the Sanger dideoxynucleotide sequencing method. Numbers along the left margin indicate the number of nucleotides from the 5′-end of the sequence. The nucleotides highlighted in bold type (313 through 1137) (SEQ. ID. NO. 1) represent the coding region of NIF1.

FIG. 3 is a schematic of NIF producing cell line construction and depicts a schematic representation of the pathway from NIF1 cDNA to the pEE14 vector which was used to transfect CHO-K1 cells.

FIG. 4 depicts the pEE14 expression vector construct employed in the construction of an NIF-expressing cell line. As indicated by its designation, the pEE14/NIF1cr expression plasmid was derived from the widely used 9.4 kb pEE14 expression vector (Lonza Biologics, Slough, UK). The pEE14 vector contains: (1) a human CMV major immediate early promoter (hCMV-MIE), (2) a multiple cloning site (MCS), (3) a SV40 early poly A site (pA), (4) a Col E1 origin of replication (Col E1), (5) an ampicillin resistance gene (Amp), and (6) the SV40 late promoter (SV40L) which drives the glutamine synthetase minigene (GS-minigene). The restriction endonuclease sites present in the multiple cloning site are noted in this diagram. The 5′ HindIII insert site is slightly 5′ to the MCS.

FIG. 5 depicts additional non-coding sequences (lower case) incorporated into the insert at both ends of the coding sequence (upper case flanking “NIF1cr”) during the cloning process (SEQ. ID. NOS. 10 and 11). The 5′-end of the insert sequences is shown to start at the HindIII site in the pEE14 expression vector (site not shown on FIG. 4), which are joined to the complementary sequences from the 5′-HindIII site from pBluescriptII shuttle vector (“BSII”) polylinker. The 5′ HindIII site is followed by an EcoRI site, provided by the 5′-PCR NIF1cr rescue primer, used to clone the NIF1cr sequences into BSII. The NIF1 coding region sequence of NIF1, beginning at this EcoRI site extends for approximately 850 nucleotides.

### DETAILED DESCRIPTION OF THE INVENTION

The process of the present invention may be carried out as described below. One of the advantages of the present invention is that it does not involve the use of animal components in any of the media, including the inoculum growth medium, the production growth medium and the nutrient feeds. This advantage is a significant in view of increasing concerns over the use of animal-derived substances in the production of medicinal drugs (e. g., fear of transmission of BSE (Bovine Spongiform Encephalopathy)). In addition, in contrast to previously-used processes using media containing animal-derived components, the process of the present invention has a processing period which is several days shorter and typically achieves appropriately glycosylated NIF titers which are 3 to 4 times greater.

Preferred Cell Lines and NIFs

The present invention is preferably practiced with mammalian cell lines, more preferably a recombinant Chinese Hamster Ovary cell line derived from CHO-K1 (ATCC CCL-61), which has been transformed with a NIF-expressing plasmid vector, preferably the pEE14 vector (Lonza Biologics; a glutamine synthetase/methionine sulfoximine co-amplification vector containing HindIII, XbaI, SmaI, SbaI, EcoRI, and BclI cloning site, wherein the vector expresses glutamine synthetase and the cloned gene) comprising NIF1 DNA (Example 1).

Construction of the NIF-producing cell line follows procedures for the establishment of cell cultures producing recombinant proteins which are known in the art and are disclosed in U.S. Pat. Nos. 5,919,900; 5,747,296; 5,789,178; 5,591,639; 5,658,759; 5,849,522; 5,122,464; 5,770,359; and 5,827,739; International Patent publication Nos. WO 87/04462; WO 89/01036; WO 86/05807 and WO 89/10404; Bebbington, et al., Bio/Technology, 10:169-175 (1992), which are all hereby incorporated by reference in their entirety.

Preferably, the cell line should be selected and adapted prior to use, such that it easily forms a suspension culture, hence is not anchorage-dependent and is weaned over several generations from animal serum and animal protein-containing media. In general, a procedure for effectuating such an adaptation may be performed by culturing the cell line analogously to that set forth in Example 2 below.

The cell line designated PFG01 (ATCC PTA-2503) is preferred for the process of the invention. The PFG01 cell line was derived from the CHO-K1 cell line (ATCC CCL-61), as set forth below in Examples 1 and 2. The PFG01 cell line was created via the transfection of the CHO-K1 cell line (ATCC CCL-61) with the pEE14 plasmid vector containing the NIF1 gene. The PFG01 cell line development was completed by generating a suspension culture from the anchorage-dependent line and weaning the recombinant cell from bovine serum.

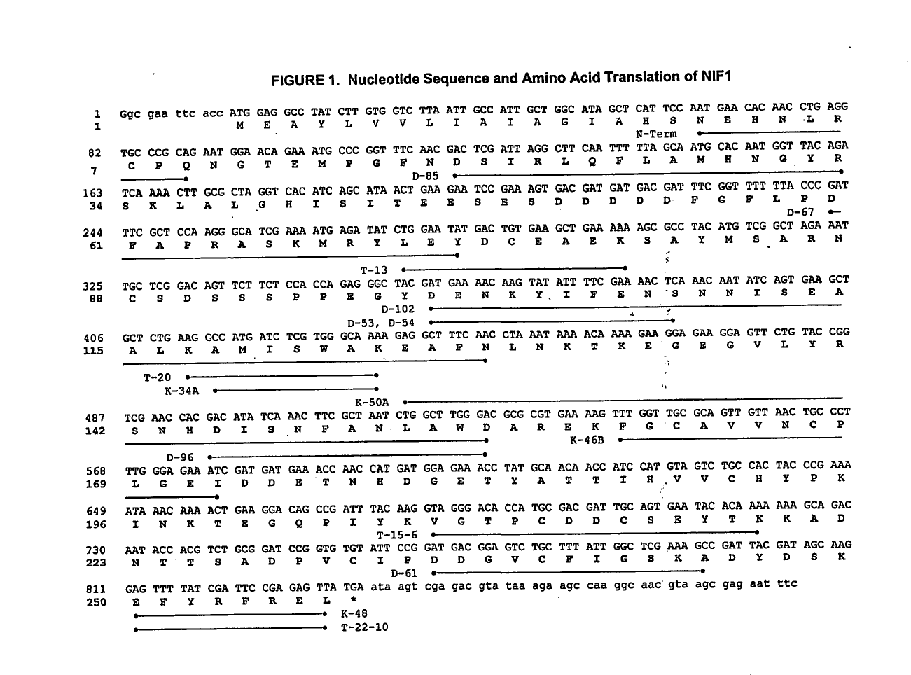

Any of the NIFs produced via cells transformed by the above-referenced methods may be produced according to the process of the present invention. Preferably, the NIF produced by the process of the present invention is a 257-amino acid protein, mature NIF-1FL (NIF1) (SEQ. ID. NO. 3) which is depicted in FIG. 1. NIF1 is produced by the transformed cells as a glycosylated and sialylated protein with a relative molecular weight of about 38.3 to about 64.1 kDa. According to a preferred aspect, NIF1 is expressed as a 41 kD glycoprotein, wherein about 30% to about 50% of its molecular weight is made up of sugar moieties (glycans) oligosaccharides, which may be branched and capped with sialic acid residues. This particular NIF is described in detail in Moyle et al., supra; see also, R. Webster et al., Xenobiotica, 29:1141-1155 (1999) and references cited therein.

Preparation of NIF

The process for preparing NIF according to the present invention involves the preparation of an inoculum via the use of an animal component-free inoculum growth medium, suspending the inoculum in a vessel containing a production growth medium, maintaining the culture of viable cells and harvesting the NIF product. In one-embodiment of the invention, the generation of the inoculum culture is conducted by growing a culture of PFG01 cells, which is then used to “inoculate” the production reactor. This inoculum culture is generated in shake flasks or in vessels, ordinarily of a size smaller than the actual production vessel.

The starting seed cells are initially suspended in a pre-warmed inoculum growth medium. If the seed cells are frozen, the seed cells expressing NIF, preferably those of the PFG01 cell line (which expires NIF1), are thawed in a bath, at a temperature of between about 30° C. and about 38° C., until the ice pellet has almost completely melted. The thawed vial is ordinarily then transferred to a bio-safe containment unit or cabinet and the exterior of the vial is decontaminated by standard means, e.g., wiping with alcohol pads, etc.

The cells are then suspended in a pre-warmed inoculum growth medium comprising:

(i) a CHO-III-PFM/glucose solution;

(ii) sodium hypoxanthine, preferably from about 50 to 100 μmol per liter (i);

(iii) thymidine, preferably from about 8 to about 32 μmol per liter (i);

(iv) an amino acid solution comprising amino acids selected from the group consisting of L-aspartic acid, L-glutamic acid, L-asparagine, L-proline, L-serine and L-methionine;

(v) optionally L-methionine sulphoximine; and

(vi) L-cysteine.

According to a preferred aspect, the inoculum growth medium comprises:

(i) a CHO-III-PFM/glucose solution, preferably Life Technologies, Custom Formula 98-0289; with 3.45 g/l D-glucose added; without hypoxanthine, thymidine, L-glutamine;

(ii) a sodium hypoxanthine/thymidine solution, preferably HT supplement (100×) (Life Technologies, Catalog No. 11067-030);

(iii) an amino acid solution, preferably composed of acids selected from the group consisting of L-aspartic acid, L-glutamic acid, L-asparagine, L-proline, L-serine, and L-methionine;

(iv) optionally an L-methionine sulphoximine (MSX) solution; and

(v) an L-cysteine solution.

Optionally, a solution containing phenol red, preferably a solution of about 0.5% w/v thereof, may be added to the media for use as a visual pH indicator. More preferably it is added in the amount of about 0 to about 3.0 ml of a 0.5% w/v solution per liter medium, most preferably about 0.5 ml per liter medium is added.

The amino acid solution, noted above, may be conveniently prepared by dissolving the amino acids in deionized water, adjusting the pH to approximately 8.0 with an aqueous base, preferably sodium hydroxide in water, followed by sterile filtering.

The MSX solution may be prepared by dissolving the MSX in deionized water and filtering the solution using a 0.2 micron filter. Aliquots of the. MSX solution may be placed into sterile tubes and may be kept for up to three months or longer at temperatures, preferably below 5° C.

More preferably the inoculum growth medium comprises:

(i) CHO-III-PFM/glucose solution (Life Technologies, Custom Formula 98-0289; with 3.45 g/l D-glucose; without hypoxanthine, thymidine, L-glutamine);

(ii) about 5 to about 20 ml per liter (i) of a 10 mM sodium hypoxanthine/1.6 mM thymidine solution;

(iii) amino acids in the noted amounts per liter (i): L-aspartic acid (about 15 to 90 mg), L-glutamic acid (about 12 to about 75 mg), L-asparagine (about 50 to about 300 mg), L-proline (about 6 to about 38 mg), L-serine (about 15 to about 90 mg) and L-methionine (about 7 to about 45 mg); more preferably the amino acids are added by adding about 5 to about 30 ml per liter (i) of an amino acid solution comprising L-aspartic acid (3.0 g/l; 22.5 mM), L-glutamic acid (2.50 g/l; 17.0 mM), L-asparagine (10.00 g/l; 75.7 mM), L-proline (1.25 g/l; 10.9 mM), L-serine (3.0 g/l; 28.5 mM), and L-methionine (1.50 g/l; 10.1 mM);

(iv) optionally about 12.5 to about 25 μmol per liter (i) L-methionine sulphoximine, if included, preferably as about 0.5 to about 3.0 ml per liter (i) of an 25 mM L-methionine sulphoximine (MSX) solution; and

(v) about 10 to about 40 mg per liter (i) of L-cysteine.

Most preferably the inoculum growth medium comprises:

(i) CHO-III-PFM/glucose solution (Life Technologies, Custom Formula 98-0289; with 3.45 g/l D-glucose; without hypoxanthine, thymidine, L-glutamine);

(ii) about 10.0 ml per liter (i) of a 10 mM sodium hypoxanthine/1.6 mM thymidine solution;

(iii) about 20.0 ml per liter (i) of an amino acid solution comprising L-aspartic acid (about 3.0 g/l), L-glutamic acid (about 2.5 g/l), L-asparagine (about 10.0 g/l), L-proline (about 1.25 g/l), L-serine (about 3.0 g/l), and L-methionine (about 1.5 g/l);

(iv) optionally about 1.0 ml per liter (i) of an 25 mM L-methionine sulphoximine (MSX) solution; and

(v) about 25.0 mg per liter (i) of L-cysteine.

The resultant inoculum growth medium is then transferred into a shake flask, or other vessel, for use in creating the inoculum, and the seed cells are suspended in it.

At initiation, the inoculum culture may be sampled and counted using, e.g., the Trypan Blue Dye Exclusion method, to determine cell concentration and viability as set forth in Cell and Tissue Culture: Laboratory Procedures in Biotechnology, A. Doyle and J. B. Griffiths, eds. (John Wiley & Sons, Ltd., 1998). If the cell concentration is greater than approximately 7.0×10⁵ viable cells per ml (“vc/ml”), more pre-warmed growth medium may be added to achieve a final concentration in the range of about 2.0×10⁵ vc/ml to about 6.0×10⁵ vc/ml, but a concentration of about 5.0×10⁵ vc/ml is preferred.

The shake flask or vessel may then be incubated with stirring at a temperature in the range of about 30 to about 38° C., preferably about 36.5±1° C.; at a CO₂ concentration of about 2 to about 10%, preferably, 5±1%; at a relative humidity of about 40 to about 90%, preferably 70±5%; and a stirring rate of about 50 to about 200 rpm, preferably 150±20 rpm (throw=⅜ inch in diameter). The flask may be sampled daily for cell concentration and viability. More pre-warmed growth medium may be added daily to maintain a concentration of about 2.0×10⁵ vc/ml to about 6.0×10⁵ vc/ml, preferably about 5.0×10⁵ vc/ml. If the volume in the vessel is exceeded by further additions of medium or the cell density reaches about 1.0×10⁶ vc/ml, the culture may be split into two or more cultures which can be diluted to about 5.0×10⁵ vc/ml in new vessels.

Subsequently, each time the cell density reaches about 1.0×10⁶ vc/ml, the culture should be split up to about 2.0×10⁵ vc/ml in further vessels. This step should be repeated in order to expand the seed train until a sufficient volume is achieved to obtain a seeding density of approximately 1.5×10⁵ vc/ml to 4.0×10⁵ vc/ml, preferably about 2.0×10⁵ vc/ml for a bioreactor vessel. The inoculum ratio (volume of inoculum culture/reactor liquid volume after inoculation) is about 10 to about 20%. The cell density in the inoculum culture should be between about 1.0×10⁶ vc/ml and about 2.5×10⁶ vc/ml, preferably between about 1.0×10⁶ vc/ml and about 1.5×10⁶ vc/ml. The age of the inoculum culture is approximately 3 to 4 days prior to use in the actual production phase.

The medium used in the actual production (production growth medium) stage for NIF differs from that used for inoculum generation. The production reactor is preferably operated under fed-batch conditions, i.e., whereby nutrient solutions are continuously fed into the reactor during the production period.

The medium for the NIF production stage (production growth medium) comprises:

(i) a CHO-III-PFM/glucose solution;

(ii) a sodium hypoxanthine;

(iii) thymidine; and

(iv) yeast extract.

Sodium hypoxanthine and thymidine may be conveniently added as a sodium hypoxanthine/thymidine solution, preferably HT supplement (100×) (Life Technologies, Catalog No. 11067-030).

Preferably the production growth medium comprises:

(i) a CHO-III-PFM/glucose solution;

(ii) sodium hypoxanthine, preferably from about 50 to 100 μmol per liter (i);

(iii) thymidine, preferably from about 8 to about 32 μmol per liter (i); and

(iv) about 0.5 to about 5 grams per liter (i) yeast extract. The CHO-III-PFM/glucose solution is preferably Life Technologies, Custom Formula 98-0289; with 3.45 g/l D-glucose; without hypoxanthine, thymidine, L-glutamine.

Optionally, phenol red, preferably a solution of about 0.5% w/v thereof, may be added for purposes of facilitating pH measurement; more preferably in the amount of about 0 to about 3.0 ml of that solution per liter of medium, most preferably, in an amount of about 0.5 ml of that solution per liter of medium.

More preferably the production growth medium comprises:

(i) CHO-III-PFM/glucose solution made by Life Technologies, Custom Formula 98-0289; with 3.45 g/l D-glucose; without hypoxanthine, thymidine, L-glutamine;

(ii) about 5 to about 20 ml per liter (i) of a 10 mM sodium hypoxanthine/1.6 mM thymidine solution; and

(iii) about 0.5 to about 5.0 grams per liter (i) yeast extract.

Most preferably the production growth medium comprises:

(i) CHO-III-PFM/glucose solution made by Life Technologies, Custom Formula 98-0289; with 3.45 g/l D-glucose; without hypoxanthine, thymidine, L-glutamine;

(ii) about 10.0 ml per liter (i) of a 10 mM sodium hypoxanthine/1.6 mM thymidine solution; and

(iii) about 1.5 grams per liter (i) yeast extract.

Preferably, two nutrient feeds are used over the course of the production stage to supply the culture with material needed for an advantageous growth rate. One of the nutrient feeds is a glucose feed (Nutrient Feed 1) at a concentration of from about 100 to about 500 g/l. This glucose feed is used to maintain the glucose concentration in the reactor at approximately 0.1 to about 5.0 g/l, preferably about 2.0 g/l. This feed is usually added at a rate of about 0.0 to about 6.0 grams of glucose per liter medium per day using a suitable pump or other means for adding the glucose spread out over time.

The second nutrient feed (Nutrient Feed 2) comprises (i) CHO-III-PFM (5-fold concentration or “5×”) solution (made by Life Technologies, Custom Formula 99-0180; with only 1× (one-fold for solubility reasons) L-cystine, 3× (three-fold for solubility reasons) L-tyrosine; without glucose, hypoxanthine, thymidine, L-glutamine, sodium bicarbonate, or sodium chloride); (ii) 25 to 100 ml per liter (i) of a 10 mM sodium hypoxanthine/1.6 mM thymidine solution; and (iii) 5 to 20 grams per liter (i) yeast extract. More preferably, the nutrient feed comprises (i) CHO-III-PFM (5×) solution made by Life Technologies, Custom Formula 99-0180 (5×) with 1× L-cystine, 3× L-tyrosine; without glucose, hypoxanthine, Thymidine, L-glutamine, sodium bicarbonate, sodium chloride; (ii) 50 ml per liter (i) of a 10 mM sodium hypoxanthine/1.6 mM thymidine solution; and (iii) 7.5 grams per liter (i) yeast extract. This second feed is prepared by adding the 10 mM hypoxanthine/1.6 mM thymidine solution, preferably HT supplement 100× (Life Technology) and the yeast extract to the CHO-III-PFM (5×) solution, dissolving and mixing together the components, adjusting the pH to about 6.8 to about 7.6 using sodium hydroxide, and then sterile filtering the final solution. This second feed solution is fed to the reactor continuously at a rate of approximately 5 to about 50 ml per liter of culture at inoculation per day starting at about 48 hours. This addition is essential for achieving high productivity of NIF with acceptable product quality.

The process of the present invention has been performed in a 2-liter Wheaton bioreactor (B. Braun Biotech Inc., Allentown, Pa.), controlled via a Foxboro IA (Intelligence Application) computer system (The Foxboro Company, Foxboro, Mass.), however any sterilizable vessel may be used as the bioreactor so long as it has an adequate mixing capability, sufficient feed inlets, two for the nutrient feeds and one for pH control, and one sampling port, is outfitted with gas inlet and purging capabilities may be used. The vessel should permit sufficient online process control. Preferably the vessel is light-impermeable or of such a nature that it may be covered to avoid direct exposure to ambient light. After sterilization of the vessel, a sterile conditioning solution may be employed to rinse out the vessel, preferably either glutamine-free DMEM (Life Technologies/GibcoBRL; Catalog No. 11960-044) or Dulbecco's Phosphate Buffered Saline (Life Technologies/GibcoBRL, Catalog No. 14190-136). After an adequate time period, depending on the size of the vessel, the rinse medium is replaced with fresh, sterile production medium. The temperature of the medium is allowed to stabilize at a temperature in the range of about 30 to about 38° C., preferably about 36.5±1° C., and if necessary the pH should be adjusted to pH about 6.8 to about 7.6, preferably a pH of about 7.4, prior to inoculation. The volume of the inoculum culture added preferably creates an initial target inoculum density in the reactor of about 1.0×10⁵ viable cells/ml to about 5.0×10⁵ viable cells/ml, preferably about 2.0×10⁵ viable cells/ml.

The contents of the vessel should be stirred at a rate in the range of about 50 to about 200 rpm, depending on the size and geometry of the vessel and the impeller used, sufficient for thorough mixing of the vessel contents. Otherwise, the contents of the vessel should be agitated in a manner which would be commensurate to achieve the same degree of mixing. The pH of the production culture should be maintained in the range of about 6.8 to about 7.6, preferably about 7.40±0.05, via appropriate control agents which do not interfere with the viability and vitality of the cell culture. In the case of the present invention, CO₂ gas and a solution of about 7.5% (w/v) NaHCO₃ is preferred as the pH control agent. However, other common alkaline solutions such as mixtures of NaHCO₃ and Na₂CO₃, or dilute NaOH may also be used successfully.

The dissolved oxygen concentration should be maintained in the range of about 10 to about 100% of air saturation, preferably about 60%±5% of air saturation via appropriate control agent. The temperature of the production culture should be maintained in the range of about 30° C. to about 38° C., preferably about 36.5° C.±1° C. The glucose concentration of the production medium preferably is maintained in the range of about 0.1 to about 5.0 g/l, preferably about 2.0 g/l±0.5 g/l, by means of a glucose feed solution (Nutrient Feed 1) which is added in small amounts at intervals to maintain the desired level.

Carbon dioxide gas and/or oxygen and/or air and/or nitrogen gas, may be sparged into the culture on demand to control the pH and dissolved oxygen. Nitrogen gas or air may be directed to the headspace to assist dissolved oxygen control and/or reduce foam generation.

The Nutrient Feed 2 is fed continuously at a rate of approximately 5 to about 50 ml per liter of culture at inoculation per day, preferably at a rate of about 25 ml per liter of culture at inoculation per day. This feed should be started simultaneously with the glucose feed, usually at about the 48 hour point.

The production culture should be sampled immediately after inoculation. The following parameters are usually measured immediately: the initial cell density and viability; the off-line pH; the initial glucose concentration; the initial lactate concentration; the initial ammonia concentration; and the initial osmolality. The on-line pH should be adjusted if necessary. The bioreactor vessel should preferably either be light-impermeable, or covered by an opaque light-blocking covering to protect the production medium from light. The production culture is usually sampled daily for the following parameters: cell density; culture viability; off-line pH; glucose concentration; lactate concentration; ammonia concentration; osmolality; and NIF concentration, purification or characterization.

The glucose concentration should be maintained between about 0.1 and about 5.0 g/liter, preferably between about 1.5 and about 2.5 g/liter using Nutrient. Feed 1. Typically, the feed begins after about 48 hours with an initial feed rate of approximately 2.0 g/(liter-day), or approximately 2.0 to about 3.0 grams glucose per 10⁹ viable cells per day, using a calibrated pump connected to an on/off timer using a 30 minute cycle. The glucose feed rate should be adjusted each day if necessary. The glucose consumption rate often changes with culture age, but the required feed rate usually remains within the range from about 0.0 to about 6.0 g/liter-day. The Nutrient Feed 2 is usually started at about 48 hours.

The process of the present invention has been carried out successfully in 2-liter stirred tank bioreactors as well as in 10-liter, 50-liter and 100-liter stirred tanks, and thus may be carried out on virtually any scale. In stirred tank reactors and using the PFG01 cell line, a NIF titer of approximately 4.0 Units/ml was reproducibly achieved in approximately eleven days. Product quality of the NIF1 produced is high based upon comparisons of post-translational sialylation/glycosylation and rat pharmacokinetic (PK) studies. PK studies of the NIF obtained by the methods of the invention may be carried out according to the protocols and techniques set forth in Webster et al., supra.

The process data for an actual 2-liter stirred tank experiment is set forth in the Examples 3 to 8. In over twenty similar reactor experiments carried out according the process of the invention, the average concentration of NIF1 produced in eleven days was approximately 4.2 Units/ml±0.4 Units/ml, as measured by the assay set forth above. Samples purified from these experiments showed reproducible post-translational modification (glycosylation/sialylation).

Determination of NIF Titer

The assay for NIF in a given sample may be conducted by HPLC chromatography, or any other means by which the concentration of NIF in a given sample may be measured.

A preferred HPLC method utilizes an HPLC column (Atlantis C5 2.0×50 mm, Phenomenex, Torrence Calif.) outfitted with a Rheodyne SS column inlet filter (0.5 μm) in line before the analytical column. Ancillary to the column are a gradient pump, a variable wavelength uv detector, an automatic sample injector with heater/cooler, a column heater, and a data collection integration system. Two mobile phases A and B are used: typically phase A is 90/10/0.05 mixture of water (J. T. Baker, HPLC grade), acetonitrile (HPLC grade) and trifluoroacetic acid (Sigma, protein sequencing grade, anhydrous) respectively; and phase B is a 90/10/0.04 mixture of acetonitrile/water/trifluoroacetic acid. These phases are prepared by stirring 900 ml and 100 ml of the 90 to 10 components, followed by filtering, degassing with stirring for several minutes, transferring to reservoir, and finally adding the trifluoroacetic acid (0.5 or 0.4 ml) with stirring for approximately 10 seconds.

Typical HPLC conditions used are: injection volume 20 μl (samples in vials in an autosampler maintained at 20° C.); uv detector at 210 nm; initial flow at 0.4 ml/min; the initial A to B ratio of 75:25; column heater set at 30° C. The typical sample injection run time is about 44 minutes under such conditions.

The pump is ordinarily set on a gradient program. A typical gradient program is as follows (Table I), although this may be adjusted according to need and setup:

TABLE I

| time | % A | % B | flow | ˜psi |
|---|---|---|---|---|
| 0 | 75 | 25 | 0.4 | |
| 20 | 48 | 52 | 0.4 | ˜600 |
| 25 | 0 | 100 | 0.8 | |
| 30 | 0 | 100 | 0.8 | |
| 35 | 75 | 25 | 0.8 | ˜1400 |
| 42 | 75 | 25 | 0.8 | |
| 43 | 75 | 25 | 0.4 | |

A standard sample of NIF is prepared from concentrate and diluted to a known concentration in PBS buffer (Dulbecco's phosphate buffered saline). Aliquots of 0.5 ml of the dilute working standard may be kept frozen. The aliquot is transferred to two autosample vials and each is injected. The peak areas of NIF are averaged. (standard concentration/average peak area=response factor). The areas of the NIF peak in assay samples are multiplied by the response factor to give the NIF concentration in the sample.

Isolation/Purification from Culture

When the NIF concentration in the production vessel has achieved a level in the range of about 1.0 to about 8.0 Units/ml NIF (or the production phase has run between about 5 and about 20 days), the NIF may then be recovered from the culture. The clarified culture fluid is obtained by centrifugation to remove cells followed by sterile filtration through an appropriate membrane, preferably a 0.22 μm filter polyethersulfone (PES) membrane. Once the filtration has been completed, the clarified culture fluid is subjected to a number of purification steps:

A. Chromatography Step 1: Q Sepharose Fast Flow Anion Exchange Chromatography

The clarified fluid containing NIF is passed through a Q Sepharose fast flow anion exchange chromatographic column whereby the NIF becomes bound to the column and is then eluted at a higher concentration salt solution. A Q Sepharose column is conditioned with 1N sodium hydroxide, followed by equilibration with 50 mM Na₂HPO₄\100 mM NaCl solution (pH 7.0). The 0.22 μm filtered culture fluid is loaded onto the column, followed by a washing with 50 mM Na₂HPO₄\100 mM NaCl solution (pH 7.0), and elution with 50 mM Na₂HPO₄\250 mM NaCl solution (pH 7.0).

B. Concentration/Diafiltration Step 1

The purified eluate from the Q Sepharose column is concentrated using a Pall 10000 MWCO Macrosep unit in a centrifuge (Sorvall RC5C Plus, HS-4 rotor, 4000 rpm, 40 minutes). During the diafiltration, the concentrated sample buffer is exchanged to 20 mM Na₂HPO₄, pH 6.0 by performing 3 cycles of buffer addition followed by centrifugation.

C. Chromatography Step 2: Phenyl Sepharose Fast Flow Hydrophobic Interaction Chromatography

A Phenyl Sepharose column is conditioned with 1N sodium hydroxide, followed by equilibration with a 20 mM Na₂HPO₄/1.0M (NH₄)₂SO₄ solution at pH 6.0. An equal volume of 20 mM Na₂HPO₄/2.0M (NH4)₂SO4 solution (pH 6.0) is added to the concentrated and diafiltered Q sepharose eluate prior to loading so that the sample is loaded in 20 mM Na₂HPO₄, 1.0M (NH₄)₂SO₄ solution (pH 6.0). The diluted diafiltrate is loaded onto the column. NIF does not bind to the column and is washed through with 20 mM Na₂HPO₄/1.0M (NH₄)₂SO₄ solution (pH 6.0).

D. Concentration/Diafiltration Step 2

The purified effluent from the Phenyl Sepharose column is concentrated and then diafiltered using a Pall 10000 MWCO Macrosep unit in a centrifuge (Sorvall RC5C Plus, HS-4 rotor, 4000 rpm, 40 minutes). During the diafiltration, the concentrated sample buffer is exchanged to 25 mM CH₃CO₂Na, pH 4.1, by performing 3 cycles of buffer addition followed by centrifugation.

E. Virus Inactivation

The pH of the flow through post diafiltration is adjusted to 3.7 with acetic acid. The sample is allowed to remain at pH 3.7 for 30 to 45 minutes with stirring, and re-adjusted to a pH of 4.1, then filtered through a Millipore 0.22 μm Steriflip filter.

F. Chromatography Step 3: DEAE Sepharose Fast Flow Anion Exchange Chromatography

In this step, NIF is bound to the column and then eluted using a solution with a higher salt concentration. The DEAE Sepharose Fast Flow Anion Exchange column is conditioned with 1N sodium hydroxide, then equilibrated with a 25 mM CH₃CO₂Na solution (pH 4.1). The sterile filtered (or DV50-filtered) material is then loaded onto the column. The column is then washed with a 25 mM CH₃CO₂Na solution (pH 4.1), and then washed with either a 25 mM CH₃CO₂Na/30 mM NaCl solution (pH 4.1) or a 25 mM CH₃CO₂Na\50 mM NaCl solution (pH 4.1), followed by elution with a 25 mM CH₃CO₂Na\300 mM NaCl solution (pH 4.1). The eluate contains the NIF product.

G. Concentration/Diafiltration Step 3

The purified eluate from the DEAE Sepharose column is concentrated and then diafiltered using a Pall 10000 MWCO Macrosep unit in a centrifuge (Sorvall RC5C Plus, HS-4 rotor, 4000 rpm, 40 minutes). During the diafiltration, the concentrated sample buffer is exchanged to 25 mM Na₂HPO₄, pH 7.0, by performing 3 cycles of buffer addition followed by centrifugation.

Measurement of Glycosylation

A protocol for the determination of the percentage of zero-, mono-, di-, tri- and tetra sialylation is described in Webster et al., Xenobiotica,. 29(11):1141-1155 (1999), which is hereby incorporated by reference.

A protocol for a determination of the total degree of sialylation of NIF is an HPLC method using PA-10 columns (Dionex Ion Pac ATC-1 mobile phase conditioner, Dionex CarboPac 4.6×50 mm PA-10 guard column and Dionex CarboPac 4.6×250 mm PA-10 analytical column) outfitted with a Dionex GP40 gradient pump, a Dionex ED40 (EC detector used in pulsed amperometric detection mode), a Dionex AS3500 autosampler and a Dionex PeakNet 5.1 software (for data acquisition and processing). The assay employs two mobile phases A (0.2M NaOH (Fisher)\50 mM sodium acetate (Sigma ACS grade) and B (0.2M NaOH\300 mM sodium acetate). Typical running conditions for the HPLC are: injection volume: 20 μl, PAD detection (optimized carbohydrate waveform), flow rate: 0.7 ml/min, initial mobile phase. A: 100% and run time: 45 minutes

The pump is ordinarily set on a gradient program. A typical gradient program (Table II) is as follows, although this may be adjusted according to need and setup:

TABLE II

| time | % A | % B | flow |
|---|---|---|---|
| 0 | 100 | 0 | 0.7 |
| 13 | 100 | 0 | 0.7 |
| 13.1 | 0 | 100 | 0.7 |
| 15 | 0 | 100 | 0.7 |
| 15.1 | 100 | 0 | 0.7 |
| 45 | 100 | 0 | 0.7 |

The purified NIF samples and a reference sialic acid standard are prepared to a concentration of about 1.0×10⁻³ Units/ml. To 200 μl aliquots of both the NIF and reference samples is added 200 μl of 0.2N HCl. The aliquots are vortexed and centrifuged briefly, then heated at 80° C. for 1 hour. The samples are then cooled in an ice bath for about 10 minutes, followed by further vortexing and centrifuging, prior to allow them to return to room temperature. A 20 μl sample is injected for analysis. Results are then reported as a percentage of the reference standard.

Determination of Neutrophil Inhibitory Activity

Assays for the determination of neutrophil inhibitory activity which may be useful in verification of the quality and biological activity of the NIF produced by the cultured cell lines are the plastic adherence assay, the calcein assay, the hydrogen peroxide release assay and ELISA set forth below.

A. The Plastic Adherence Assay

i. Isolation of Neutrophils

Neutrophils are isolated from heparinized venous blood using a one-step Ficoll-Hypaque gradient (Mono-poly, ICN Biomedicals, Irvine, Calif.). Briefly, 5 ml whole blood is layered onto 3 ml of Mono-poly resolving media in a 16×100 mm glass tube. Separation of leukocytes is achieved by centrifuging at 300×g for 60 minutes at 20° C. The layer of cells containing neutrophils was collected using a Pasteur pipette and cells were suspended in 10 volumes of cold Delbeccos' modified Eagle's medium (DMEM, Life Technologies, Gaithersburg Md.). Neutrophils were pelleted at 200×g for 10 minutes at 4° C. The cell pellet was resuspended in 5 ml cold ACK buffer (155 mM NH₄Cl/10 mM KHCO₃, pH 7.4) and incubated for 5 minutes at room temperature to lyse contaminating red blood cells. Neutrophils were then washed once by centrifugation and resuspended in HBSS (1.33 mM CaCl₂, 0.5 mM MgCl₂, 0.04 mM MgSO₄, 140 mM NaCl, 5 mM KCl, 0.3 mM KH₂PO₄, 0.3 mM Na₂HPO₄ with 5.6 mM D-glucose and 30 mg/l phenol red) at an approximate concentration of 10⁷ cells/ml. Cell viability was determined by Tryptan blue exclusion. These preparations were consistently greater than 95% neutrophils as determined by automated differential counting.

ii. The Plastic Adherence Assay

Stimulated human neutrophils will adhere to plastic tissue culture ware and can be visualized by standard phase contrast light microscopy. Neutrophils, isolated as in step A above, are washed once and resuspended in cold HSA buffer (RPMI without sodium phosphate (Life Technologies), 1% human serum albumin (Calbiochem, San Diego, Calif.), 1.2 mM CaCl₂, 1.0 mM MgCl₂, 10 mM HEPES, pH 7.3) at a concentration of 6.6×10⁶ cell/ml. Neutrophils (20 μl) are placed in a sterile microfuge tube and stimulated with PMA (5 μl of a 800 nM solution or 160 nM final concentration) for 5 minutes at 37° C. The sample to be tested is added (20 μl) to tube containing the stimulated cells, mixed gently, and 10 μl of the mixture is immediately transferred to each well of a Terasakistyle culture plate (Nalge Nunc International, Naperville, Ill.). After an additional 5 minutes at 37° C., the entire plate is immersed in Hanks' balanced salt solution (JRH Biosciences, Lenexa, Kans.) and tapped to dislodge non-adherent cells. The tap/rinse step is repeated a total of six times. Cells adhered to the plastic wells are visualized using a phase contrast light microscope. Control wells with stimulated cells and no test sample are scored “++++”, control wells with stimulated cells and the monoclonal antibody CLB-54 (directed against the integrin CD11b/CD18) are scored

B. Neutrophil-Huvec Adherence (Calcein) Assay

The adherence of human neutrophils to HUVEC monolayers are monitored by using cells which are preloaded with the fluorescent dye calcein-AM (acetoxymethyl ester; Molecular Probes, Eugene, Oreg.). Human neutrophils are labeled with calcein as follows. Neutrophils are pelleted and resuspended in HBSS containing 10 μg/ml calcein-AM at a final cell concentration of approximately 10⁷ cells/ml. The working HBSS/calcein solution is prepared immediately before use from a stock solution of calcein in dimethylsulfoxide (10 mg/ml, stored at −20° C.). Neutrophils are incubated with calcein-AM for 30 minutes at 37° C. with intermittent mixing every 10 minutes. Labeled neutrophils are washed once and resuspended in cold HSA buffer (RPMI without sodium phosphate (Life Technologies), 1% human serum albumin (Calbiochem, San Diego, Calif.), 1.2 mM CaCl₂, 1.0 mM MgCl₂, 10 mM HEPES, pH 7.3.) at a concentration of 1.32×10⁷ cell/ml. Cells are kept at 4° C. until used.

Calcein-labeled neutrophils (175 μl) are incubated for 10 minutes at 20° C. with a 175 μl test fraction in the presence of 100 ng/ml PMA (Sigma, St. Louis, Mo.). A stock solution of 1 mg/ml PMA was prepared in dimethyl sulfoxide and routinely stored at −70° C. One hundred μl of the test fraction/PMA-treated cells (6.6×10⁵ neutrophils) are added to a confluent monolayer of primary HUVECs (Clonetics, San Diego, Calif.) grown in a 96-well microtiter plate (Costar, Cambridge, Mass.). After 30 minutes at 37° C., non-adherent cells are removed by centrifuging inverted, sealed plates for 3 minutes at 75×g. Adherent neutrophils were lysed by adding 100 μl 0.1 Triton X-100 (in 50 mM Tris-HCl, pH 7.4) and the fluorescent emission of calcein at 530 nm from 485 nm excitation is reading using a Cytofluor fluorometric plate reader (Millipore, Bedford, Mass.). Each data point is performed in triplicate. In these experiments, 40% of the total input neutrophils, or approximately 2.6×10⁵ cells, bind to the HUVEC monolayer in the absence of inhibitor.

C. Hydrogen Peroxide Release Assay

Hydrogen peroxide release from stimulated human neutrophils is determined by a modification of the method described by Pick et al., J. Immun. Methods, 38:161-170 (1980). Human neutrophils (6.6×10⁶ cell/ml) are resuspended in HBSS containing 10% fetal bovine serum. Phenol red and Type IV horseradish peroxidase (Sigma) are added to the cell suspension at final concentrations of 83 μg/ml and 0.01 Units/ml, respectively. Five hundred microliters of this cell suspension are added to 200 μl of test sample (in HBSS containing 10% fetal bovine serum). Cells are activated with fMLP (Sigma) at a final concentration of 275 μM. A stock solution of fMLP (500 nM) is prepared in dimethyl sulfoxide and stored at −20° C. The release assay is performed in 1.5 ml plastic tubes (Eppendorf, Madison, Wis.) that are precoated with fetal bovine serum for 60 minutes at 37° C.; coated tubes are washed twice with 0.15N NaCl before use. The release action is allowed to proceed for 90 minutes at 37° C., after which time the cells are pelleted at 2000×g for 3 minutes in an Eppendorf Microfuge. Two hundred microliters of supernatant fluid are transferred to a 96-well microtiter plate and the reaction is stopped by the addition of 10 μl of 1N NaOH. Each data point is performed in duplicate. Samples are quantitated at 610 nm with a ThermoMax plate reader (Molecular Devices, Sunnyvale, Calif.). Hydrogen peroxide concentration was calculated from an internal standard curve.

D. ELISA for NIF1

A polyclonal antibody directed against NIF1 is prepared in rabbits using standard techniques. The antibody is immunoaffinity-purified using resin composed of rNIF1 coupled to uniform glass beads (Bioprocessing Ltd., Consett, UK). A monoclonal antibody directed against NIF1 is also prepared in mice using standard techniques. The monoclonal antibody is purified from mouse ascites fluid by protein A chromatography and conjugated to horseradish peroxidase (“HRP”) (Boehringer Mannheim, Indianapolis, Ind.) following standard protocols. The immunoaffinity-purified polyclonal antibody is adsorbed to the wells of Immulon 2 polystryrene immunoassay plates (Dynatech Labs, Chantilly, Va.) and then blocked with bovine serum albumin. Test samples containing NIF1 (100 μl/well) are added to the wells of the immunoassay plate, mixed using a plate shaker, and incubated at 37° C. for 3 hours. The contents of the wells are removed and the wells washed with phosphate buffered saline containing 0.02% Tween 20. Monoclonal antibody-HRP conjugate (100 μl/well) is added to the wells, mixed as before, and incubated at 37° C. for 2 hours. Unadsorbed monoclonal antibody-HRP conjugate is rinsed away with phosphate buffered saline containing 0.02% Tween 20 and HRP substrate (10 ml of 0.1M sodium acetate, pH 4.5, 0.012% hydrogen peroxide, plus 0.4 ml trimethylbenzidine, 3 mg/ml in 0.1M HCl) was added to the wells. Color is allowed to develop for 10 minutes at room temperature when the reaction is stopped with 1M sulfuric acid. The optical density at 450 nm is determined using a Molecular Devices 96-well plate reader. Standard curves are generated with samples containing known concentrations.

Formulations

Pharmaceutical compositions of NIF may be formulated and used as tablets, capsules or elixirs for oral administration; suppositories for rectal administration; sterile solutions, suspensions for injectable administration; and the like. The dose and method of administration can be tailored to achieve optimal efficacy but will depend on such factors as weight, diet, concurrent medication and other factors which those skilled in the medical arts will recognize. Generally, an amount between 0.01 mg/kg to 100 mg/kg body weight/day is administered dependent upon the potency of the composition used. Preferred embodiments encompass pharmaceutical compositions prepared for storage and subsequent administration which comprise a therapeutically effective amount of NIF or an enriched composition of NIF, as described herein in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro, Ed. 1985). Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride or the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes) may be utilized.

Utility

The NIF produced in the present invention may be used in methods of treating in a mammal an inflammatory condition characterized by abnormal neutrophil activation or abnormal eosinophil activation comprising administering to said mammal a therapeutically effective amount of a NIF or their pharmaceutical compositions. In practicing the preferred methods, NIFs or their pharmaceutical compositions can be used alone or in combination with one another, or in combination with other therapeutic or diagnostic agents. These compositions can be utilized in vivo, ordinarily in a mammal, preferably in a human, or in vitro.

In employing NIFs or their pharmaceutical compositions in vivo, the compositions can be administered to the mammal in a variety of ways, including parenterally, intravenously, subcutaneously, intramuscularly, colonically, rectally, nasally or intraperitoneally, employing a variety of dosage forms. As will be readily apparent to one skilled in the art, the useful in vivo dosage to be administered and the particular mode of administration will vary depending upon the mammalian species treated, the particular composition employed, and the specific use for which these compositions are employed. The determination of effective dosage levels, that is the dosage levels necessary to achieve the desired result, will be within the ambit of one skilled in the art. Typically, applications of compositions are commenced at lower dosage levels, with dosage level being increased until the desired effect is achieved.

The dosage for a NIF or its pharmaceutical compositions can range broadly depending upon the desired effects and the therapeutic indication. Typically, suitable dosages will be between about 0.01 mg and about 100 mg/kg, preferably between about 0.01 and about 10 mg/kg, body weight. Administration is preferably parenteral, such as intravenous on a daily or as-needed basis.

The NIF produced by the methods of the present invention has potent neutrophil inhibitory activity and, thus, may be used as an inhibitor of neutrophil activity, including neutrophil activation in vitro, as well as for preventing or treating in a mammal inflammatory conditions characterized by abnormal neutrophil activation. Thus, NIF will be useful in the treatment of inflammation in which the abnormal activation of neutrophils plays a significant role. While applicants do not wish to be bound to any theory or mode of activity, it is believed that this compound will interfere with the inflammatory response which is set into action by neutrophil-endothelial cell interactions. Thus, where adhesion of neutrophils to the endothelium is prevented, the neutrophils will be unable to transmigrate to tissue to elicit a pro-inflammatory response with consequent tissue damage. Inhibition of neutrophil-neutrophil adhesion and/or aggregation by these NIFs should also prevent microvascular occlusion. Thus, these NIFs will be useful in treating a variety of clinical disorders, including shock, stroke, acute and chronic allograft rejection, vasculitis, autoimmune diabetes, rheumatoid arthritis, head trauma, inflammatory skin diseases, inflammatory bowel disease, adult respiratory distress syndrome (ARDS), ischemia-reperfusion injury following myocardial infarction, in which neutrophil infiltration and activation has been implicated and acute inflammation caused by bacterial infection, such as sepsis or bacterial meningitis.

The ability of the NIF produced by the present invention to inhibit neutrophil activity makes it useful in inhibiting the physiological processes of inflammation, ischemia, and other neutrophil mediated tissue damage. The specific activities of NIFs in carrying out these related functions makes it particularly useful as therapeutic and/or diagnostic agents.

Antibodies, both monoclonal and polyclonal, directed to the NIF produced by the present invention are useful for diagnostic purposes and for the identification of concentration levels of the subject peptides in various biological fluids. Immunoassays utilizing these antibodies may be used as a diagnostic test, such as to detect infection of a mammalian host by a parasitic worm or to detect NIF from a parasitic worm in a tissue of the mammalian host. Also such immunoassays may be used in the detection and isolation of NIF from tissue homogenates, cloned cells and the like. In another aspect of the present invention, NIFs can be used in a test method to screen other compounds to detect NIF mimics or to detect NIF antagonists for their ability to affect NIF binding to the CD11b/CD18 receptor.

In yet another aspect of the present invention, the NIF produced by the present invention with suitable adjuvants can be used as a vaccine against parasitic worm infections in mammals. Immunization with NIF vaccine may be used in both the prophylaxis and therapy of parasitic infections. NIF fragments and synthetic polypeptides having the amino acid sequence of NIF may also be used as vaccines. Disease conditions caused by parasitic worms may be treated by administering to an animal infested with these parasites substances which antagonize NIF (such as NIF antagonists). Compounds may be screened for their anti-NIF effect according to the screening method described herein above. Examples of such antihelminic agents include antibodies to NIF, both naturally occurring antibodies isolated from serum and polyclonal and monoclonal antibodies described above. Chemically synthesized compounds which act as inhibitors of NIF also are suitable antihelminic agents.

The following examples serve to illustrate the process of the invention. The actual allowed ranges for process control parameters and process scale may be significantly broader.

### EXAMPLES

### Example 1

Cell Line Expressing NIF

A. The Nucleic Acid Encoding NIF

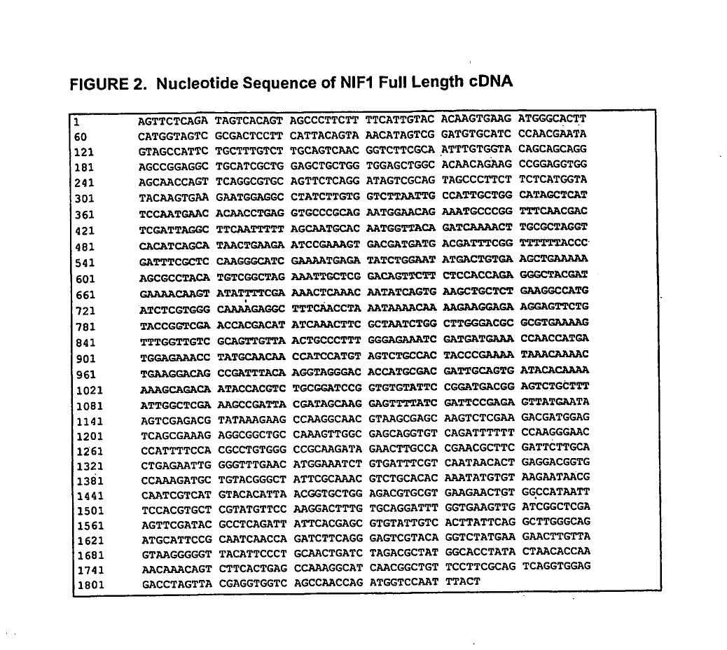

The coding sequence for recombinant NIF was derived from a canine hookworm (Ancylostoma) cDNA library to which standard expression regulatory sequences were added during plasmid construction. The nucleotide sequence of NIF-1FL, mature NIF-1FL (NIF1) and the corresponding full-length cDNA are presented in FIGS. 1 and 2, respectively. The nucleotide sequence in FIG. 2 has an open reading frame of 822 nucleotides encoding a 274 amino acid polypeptide (nucleotides 313 through 1134).

B. Construction of the Expression Vector

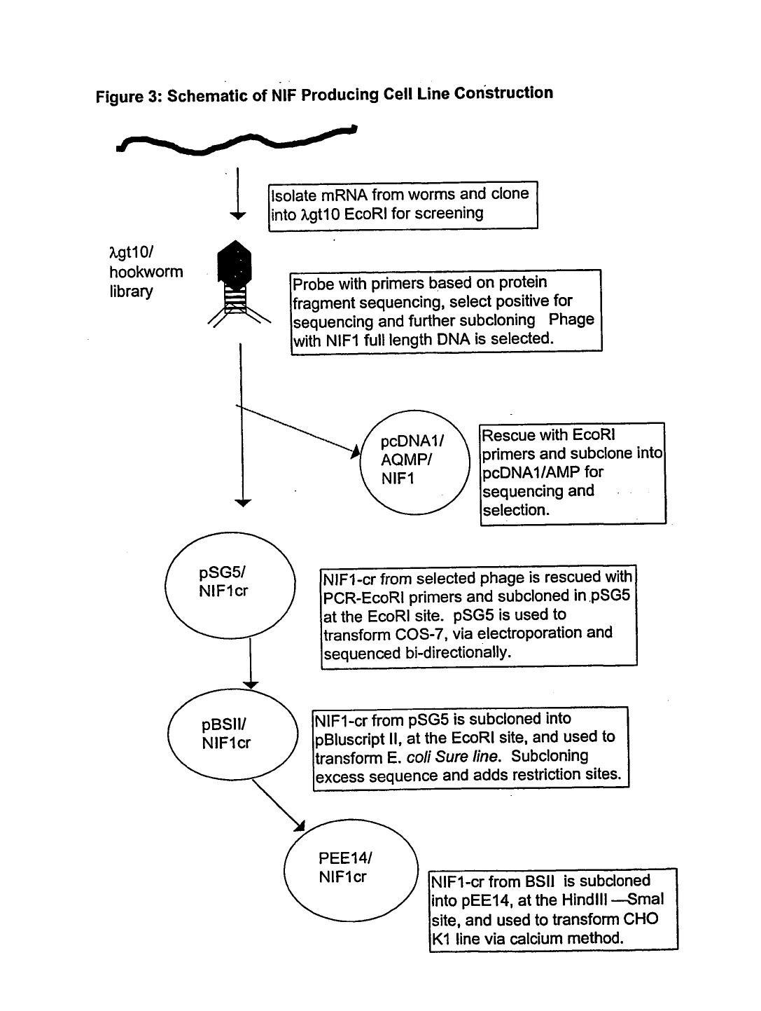

The NIF1 cDNA described above was cloned into a series of shuttle vectors and hosts, and finally into the pEE14 vector, as follows. FIG. 3 is a schematic representation of the pathway from NIF1 cDNA to pEE14 vector which was used for transfecting CHO-K1 cells. Some of the biochemicals used in the cell line construction process and their respective suppliers are as follows (Table III):

TABLE III

| Vector | Supplier |
|---|---|
| Lambda gt10/EcoRI | Stratagene, La Jolla, |
| | California |
| pcDNA1/Amp (sequencing | Invitrogen, Carlsbad, |
| vector, not used in | California |
| cloning) | |
| pBluescript II KS+ | Stratagene, La Jolla, |
| | California |
| pSG5 | Stratagene, La Jolla, |
| | California |
| E. coli SURE ™ | Stratagene, La Jolla, |
| | California |

The NIF1 coding region (“NIF1cr”) (SEQ. ID. NO. 1) was rescued into the pSG5 vector and the nucleotide sequence determined. The coding region itself (disregarding the sequence immediately upstream from the ATG start which was altered to promote expression) corresponds exactly with bases 313 through 1137 of the original NIF1 cDNA clone as illustrated in FIG. 2.

COS-7 cells transfected with pSG5/NIF1cr produced active NIF1. The NIF1 produced inhibited H₂O₂ production by human neutrophils in a concentration dependent manner as does hookworm-derived NIF. Neither control transfection (cells transfected with a plasmid harboring chloramphenicol acetyl transferase or mock transfected cells) produced a NIF-like activity.

The limited multiple cloning site of pSG5 necessitated the passage of NIF1cr through a plasmid capable of supplying different restriction sites on opposite ends of the coding region. The pBluescript II KS+ was selected due to the presence of an EcoRI site in the middle of its extensive multiple cloning site and its ease of manipulation. This was followed by cloning into the pEE14 expression plasmid. The expression construct pEE14/NIF1cr was used to transfect CHO K1 cells.

Proper isolation and sequence of final and intermediate constructions was verified at critical steps by restriction mapping or sequencing. The full length NIF sequence was first verified by sequencing when cloned into the pcDNA1/Amp sequencing vector (see, FIG. 2 for sequence). The coding region sequenced was verified after cloning into the PSG5 shuttle vector by bi-directional sequencing.

C. The Expression Vector

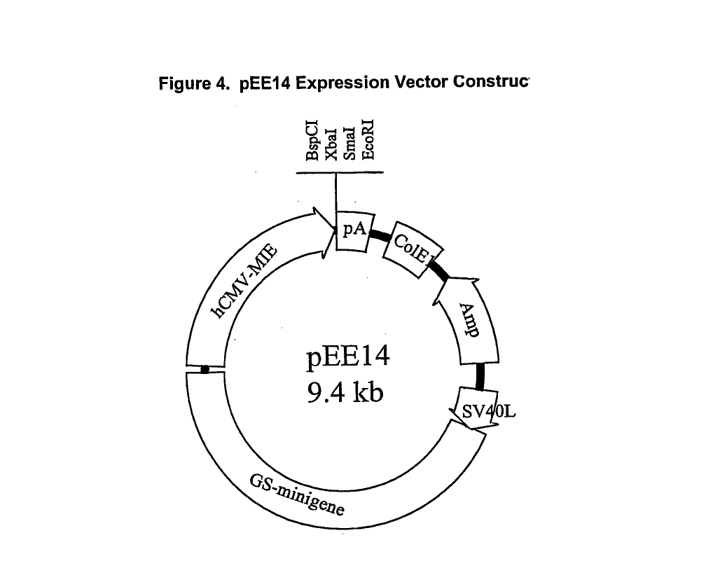

As indicated by its designation, the pEE14/NIF1cr expression plasmid was derived from the widely used 9.4 kb pEE14 expression vector (Lonza Biologics) shown at FIG. 4. The pEE14 vector contains: (1) a human CMV major immediate early promoter (hCMV-MIE), (2) a multiple cloning site (MCS), (3) a SV40 early poly A site (pA), (4) a Col E1 origin of replication (Col E1), (5) an ampicillin resistance gene (Amp), and (6) the SV40 late promoter (SV40L) which drives the glutamine synthetase minigene (GS-minigene). The restriction endonuclease sites present in the multiple cloning site are noted in this diagram. The 5′ HindIII insert site is slightly 5′ to the MCS. Nucleotide sequences for portions of the vector can be obtained from Bebbington et al., Bio/Technology, 10:169-175 (1992) and Stephens and Cockett, Nucleic Acids Research, 17:7110 (1989).

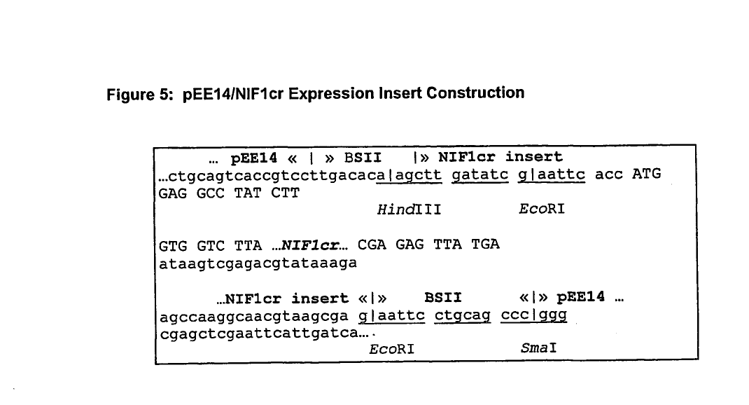

The pEE14/NIF1cr insert contains 825 bp of NIF1 coding sequence (SEQ. ID. NO. 1), which codes for the 274 amino acids indicated in FIG. 1 (SEQ. ID. NO. 2). The mature NIF-1FL (NIF1) protein contains the 257 amino acids coded by the sequences starting with codon 18, as indicated in FIG. 1 (SEQ. ID. NO. 3). Additional non-coding sequences (SEQ. ID. NOS. 10 and 11) were incorporated into the insert, at both ends of the coding sequence during the cloning process, as shown in FIG. 5.

The principal modification to the pEE14 vector is the insertion of the NIF1 coding sequence into the vector's insert expression region between the pEE14 HindIII (bp9292) and SmaI sites (bp9334). This construction allows for high level NIF1 expression under the control of pEE14's human CMV major intermediate early (“hCMV-MIE”) promoter. The construction of NIF1cr (cr=coding region) insert is depicted in FIG. 5.

As shown in FIG. 5, the 5′-end of the insert sequences start at the HindIII site in the pEE14 expression vector (site not shown on FIG. 4), which are joined to the complementary sequences from the 5′-HindIII site from pBluescriptII shuttle vector (“BSII”) polylinker. The 5′ HindIII site is followed by an EcoRI site, provided by the 5′-PCR NIF1cr rescue primer, used to clone the NIF1cr sequences into BSII. The NIF1 coding region sequence of NIF1, beginning at this EcoRI site extends for approximately 850 nucleotides.

The NIF1cr is followed by the EcoRI site created by the 3′-PCR NIF1 rescue primer used to create 3′ end needed for cloning NIF1cr into the BSII. The 3′-end of the coding region is followed by a PstI site from pBluescriptII shuttle vector, and finally a SmaI site and other sequence from pEE14 (FIGS. 4 and 5). The NIF1 protein coding sequences are shown in capitals, and bars indicate the cleavage points for the indicated restriction enzymes. The pEE14 sequences between the HindIII and SmaI sites are removed during NIF1cr cloning.

D. Transfection of the Expression Vector

The pEE14/NIF1cr vector was introduced in CHO-K1 cells (ATCC CCL-61) using a standard calcium method as follows. For transformation, the CHO-K1 cells were propagated in DMEM (Life Technologies/Gibco) in T-75 flasks at 37° C. in a 7.5-10% CO₂ atmosphere. To each 500 ml DMEM was added: standard nutrients and 50 ml fetal bovine serum (FBS). Prior to transfection, the cells were removed from the flasks using porcine trypsin as described above and washed with DMEM-S (DMEM prepared as above but with dialyzed FBS) and seeded onto 10 cm diameter tissue culture plates (Costar) at 1×10⁶ cells per plate. The cells were incubated at 37° C. overnight. Just before the cells were to be transformed, they were rinsed once with DMEM without FBS. The DNA-calcium phosphate precipitate was prepared in two steps as follows. First 62 μl 2M calcium chloride was mixed with 10 μg pEE14/NIF1cr DNA and brought up to 500 μL with sterile water. Next this mixture was added dropwise to 500 μl 2×HEPES buffered saline with constant gentle agitation using a bubble stream. Once all of the DNA mix was added the tube containing the DNA-calcium phosphate precipitate was vortexed. The DNA-calcium phosphate precipitate was diluted with 2 ml DMEM without FBS and added to the 10 cm diameter dish containing the CHO K1 cells. The plates were placed at 37° C., 7.5% CO₂ with gentle rocking for 4 hours. The medium and DNA-calcium phosphate precipitate was removed from the cells and replaced with 3 ml 15% glycerol in HEPES buffered saline. After 90 seconds at 37° C., 10 ml DMEM without FBS was added and immediately removed by aspiration. The cells were then covered with 10 ml DMEM-S and incubated for 24 hours at 37° C., 7.5% CO₂. The medium was replaced with fresh DMEM-S containing 25 μM methionine sulfoximine (MSX). The plates were incubated for an additional 7 days when the CO₂ was raised to 10% to lower the pH of the medium. At this time the plates contained many colonies of various sizes. The cells were removed from the dishes by treatment with porcine trypsin as before, collected by centrifuging as before, and resuspended in 50 ml of an equal volume mixture of conditioned medium and fresh DMEM-S supplemented with 20% dialyzed FBS and 25 μM MSX. The resuspended cells were transferred into 96-well culture plates (100 μl per well) and incubated at 37° C., 10% CO₂ to obtain individual colonies. Seven days after plating, 100 μl cloning medium (50% CHO K1 conditioned DMEM-S with 20% dialyzed FBS, 50% fresh DMEM-S with 20% dialyzed FBS, 25 μMSX) was added to replace medium lost to evaporation. Ten days after plating, 201 wells contained individual colonies and 3 wells contained 2 or 3 colonies.

Twenty days after plating, 15 wells exhibited confluent growth and the cell-free supernatant fluids were assayed using the plastic adhesion assay (above) for NIF (and, thus, NIF1) activity. The positive clones were expanded into 24-well culture plates as follows. The cells were detached from the 96-well plates by treatment with porcine trypsin and the digestion with trypsin stopped with trypsin inhibitor. One ml of cloning medium was added to each well and the plate was incubated at 37° C., 10% CO₂. Three days later 500 μl cloning medium containing 25 μM MSX was added. Seven days after expansion, cells were assayed for NIF1 activity using the plastic adhesion and calcein assays. Positive clones were expanded into 10 cm diameter tissue culture dishes. One clone expressing the highest level of NIF1 activity was frozen at approximately 1×10⁶ cell/ml in cloning medium containing 20% dialyzed FBS, 25 μM MSX and 10% dimethyl sulfoxide.

This clone was subjected to two rounds of cloning by limited dilution to ensure the final cell line originated from a single transfected cell. The clone, grown to confluence in the DMEM-S containing 10% dialyzed FBS and 25 μM MSX, was removed from a culture dish by trypsin treatment, diluted with cloning medium to 25 cells per ml, and plated in 96-well plates at 2.5 cells per well. The plates were incubated at 37° C., 10% CO₂. After 17 days in culture, 33 of the wells (those exhibiting growth) were assayed for NIF activity using the calcein assay. On the basis of growth rate and expression of NIF activity, several of the clones were grown to confluence in DMEM-S containing 10% dialyzed FBS and 25 μM MSX and frozen at approximately 1×10⁶ cell/ml in cloning medium. All cultures were confirmed to be producing NIF1 by ELISA (see Detailed Description of the Invention).

### Example 2

Adaptation to Suspension Culture and Serum-Free Medium

A. Subculturing of Cell Line in T-Flask Cultures

One of the cultures as prepared in Example 1 was further grown in a medium consisting of DMEM:RPMI1640 50:50 (glutamine free) (50:50 mix of DMEM (Dulbecco's Modified Eagle Medium, Gibco Catalog No. 11960) and RPMI1640 (Roswell Park Memorial Institute, Gibco Catalog No. 21870); 10% Certified Heat Inactivated Fetal Bovine Serum (Gibco); with 1 ml per liter medium of a 25 mM (1000×) L-methionine sulfoximine stock solution (Sigma).

The medium was decanted off. The monolayer was rinsed twice with 10 ml of Dulbecco's PBS (calcium and magnesium free); the Dulbecco's PBS was decanted and 2 ml of versene was added to the monolayer. The culture with versene was incubated at 37° C. for 5 minutes. The flask was rapped several times to dislodge the cells and resuspended in an additional 18 ml of fresh medium and split 1:5 to new T-flasks. The culture was incubated at 37° C. in 5% CO₂ and 70% humidity and designated as passage X+1. A solution of 0.25% trypsin EDTA was used in the place of versene for all subsequent subcultures in T-flasks. Cultures were typically split 1:10 to 1:25 as necessary twice per week. Cells were not allowed to reach 100% confluence if possible.

B. Adaptation of Culture to Suspension Growth

The culture was adapted to suspension growth in shake flasks in CHO III PFM medium supplemented with serum. The suspension culture was inoculated with cells from T-flasks at passage X+4. The suspension medium formulation consisted of CHO III PFM (Gibco Formula # 96-03345A); 10% Certified Heat Inactivated Fetal Bovine Serum (Gibco 10082); 1 ml/l of a 25 mM (100×) L-methionine sulfoximine solution; and 10 ml/l 100×HT supplement (Gibco 11067).

The suspension culture was inoculated at a density of 1.7×10⁵ cells/ml. The medium volume was 50 ml in a 250 ml Corning disposable shake flask. The culture was incubated at 37° C. with 5% CO₂ and 70% humidity on a shaker at 130 rpm. The culture was split 1:3 to 1:5 as needed when the cell density approached 1×10⁶ cells/ml and was never split to a density below 2×10⁵ cells/ml.

The first passage of the cells in suspension shake flask culture was designated passage X (X+5 from T-flasks). The culture was continued out to passage X+7 with 10% fetal bovine serum. The adaptation to suspension growth in serum supplemented medium took approximately 20 days.

C. Adaptation to Serum-Free Suspension Growth

The suspension culture was adapted to serum free growth by gradually decreasing the concentration of serum in the medium. All other components of the medium formulation were unchanged during the weaning process. As with the suspension growth adaptation, cells were maintained between 2.5×10⁵ and 1×10⁶ cells/ml by splitting 1:3 to 1:5 as necessary. The culture was incubated at 37° C. with 5% CO₂ and 70% humidity on a shaker at 130 rpm. At passage 8, the serum was reduced to 5%; at passage 9, to 2%; at passage 10, to 1%; at passage 11, the cells were centrifuged and resuspended in 40 ml fresh medium/10 ml conditioned medium, serum concentration was maintained at 1% (large clumps and cell debris were allowed to settle from the culture and removed); at passages 12-15, serum was maintained at 1%; at passage 15, the medium was supplemented with 60 mg/l L-aspartic acid, 120 mg/l L-serine, 200 mg/l L-asparagine and 60 mg/l L-methionine added as a 50× stock solution adjusted to pH 7.5 and filter sterilized; at passages 16-21, the serum was reduced to 0% and the amino acid supplements were maintained; and at passage 21, a pre-seed stock (PSS) frozen vial bank was prepared at 1×10⁷ cells/vial. Adaptation to serum free medium took approximately 55 days. The serum-free suspension culture as produced herein was designated PGF01.

D. Freezing the Culture

The cells as prepared above were prepared for freezing and storage by centrifuging the cells at 10 minutes at 500 rpm in a Beckman GPR centrifuge. The cells were resuspended in freezing medium consisting of 50% complete medium (CHO-III-PFM with 1 ml/l 25 mM (1000×) methionine sulfoximine stock solution, 10 ml/l HT supplement, 20 ml/l 50× amino acid stock solution, 3 g/l L-aspartic acid, 6 g/l L-serine, 10 g/l L-asparagine, 3 g/l L-methionine); 50% conditioned medium. “Conditioned” medium is one in which the cells have been grown for a few days, the cells centrifuged and separated out, and then filter sterilized; 10 g/l bovine serum albumin as a protectant; and 75 ml/l DMSO (Sigma Cell Culture Tested D2650) to a density of 1×10⁷ cells/ml and dispensed in cryovials. The cells were frozen in a controlled rate freezer to −75° C. at 1° C./minute and then transferred to liquid nitrogen for storage.

E. Preparation of Serum-Free Suspension Master Cell Bank (MCB)

A master cell bank was prepared from the Passage X+21 cell culture removing one vial of the passage X+21 culture from liquid nitrogen storage and quickly thawed in a 37° C. water bath. The contents of the vial was transferred to a 15 ml conical tube with 9 ml of fresh medium consisting of CHO III PFM (Gibco Formula # 96-03345A); 1 ml/l of a 25 mM (1000×) L-methionine sulfoximine stock solution; 10 ml/l 100×HT supplement (Gibco 11067); 20 ml/l 50× amino acid stock solution adjusted to pH 7.5 and filter sterilized (stock solution: 3 g/l L-aspartic acid, 6 g/l L-serine; 10 g/l L-asparagine and 3 g/l L-methionine

The tube was centrifuged at 500 rpm for 10 minutes in a Beckman GPR centrifuge. The supernatant was decanted and the cells were dislodged from the bottom of the tube. Ten ml of fresh medium was added to the tube and the suspended cells were transferred to a Corning 250 ml shake flask. The medium volume was adjusted to 50 ml making the initial cell density in the culture 2×10⁵ cells/ml. The culture was incubated overnight at 37° C. in 5% CO₂ with 70% humidity on a rotary shaker at 100 rpm. After the first day of incubation, the rotary shaker speed was increased to 130 rpm. After 5 passages in serum free/animal protein free suspension growth, an MCB was prepared at 1×10⁷ cells/vial.

### Example 3

A. Medium for the Generation of the Inoculum Culture

The culture medium for the inoculum culture was prepared from the following components:

1.0 liter CHO-III-PFM solution with glucose (Life Technologies, Custom Formula 98-0289 ; with 3.45 g/l D-glucose; without hypoxanthine, thymidine, L-glutamine);

10.00 ml/l HT supplement (Life Technologies, Catalog No. 11067-030; 100×=10 mM sodium hypoxanthine, 1.6 mM thymidine);

20.00 ml/l amino acid stock (as prepared in 3B below);

1.00 ml/l 25 mM L-methionine sulphoximine stock (as prepared in 3C below);

25.00 mg/l L-cysteine (Sigma); and

0.50 ml/l phenol red (Sigma, 0.5% (w/v) solution).

B. Amino Acid Stock

The amino acid stock used in the inoculum culture medium above was prepared by dissolving: 3.00 g/l L-aspartic acid (Sigma), 2.50 g/l L-glutamic acid (Sigma), 10.00 g/l L-asparagine (Sigma), 1.25 g/l L-proline (Sigma), 3.00 g/l L-serine (Sigma), and 1.50 g/l L-methionine (Sigma) in deionized water to make a one liter solution, adjusting the pH to 8.0 with aqueous 5N sodium hydroxide and then sterile filtering the resultant solution.

C. L-Methionine Sulphoximine Stock

L-methionine sulfoximine (25 mmol, FW 180.2, Sigma) was dissolved in one liter of deionized water. The resultant solution was filtered using a 0.2 micron filter. This solution may be kept at 4° C. for up to 3 months, or can be stored frozen at −20° C. or lower for longer periods of time.

### Example 4

Inoculum Generation

A vial of frozen PFG01 seed cells were thawed in a water bath at 36.5±1° C. until only a small ice pellet remained. The vial was transferred to the biosafety cabinet and the exterior decontaminated with a sterile, 70% isopropanol wipe. The cells were resuspended in 25 ml of pre-warmed growth medium as prepared in Example 3A and transferred into a 125 ml shake flask. The culture was sampled using the Trypan Blue Dye Exclusion method (Cell and Tissue Culture: Laboratory Procedures in Biotechnology, A. Doyle and J. B. Griffiths, eds. (John Wiley & Sons, Ltd., 1998)). If necessary, more pre-warmed growth medium was added to adjust the final cell concentration to approximately 5.0×10⁵ vc/ml. The flask was incubated with stirring at 36.5±1° C., CO₂ concentration of 5±1%, a relative humidity of 70±5%, and a stirring rate of 170±5 rpm. The flask was sampled daily to check cell concentration and viability.

Sufficient pre-warmed growth medium was added daily to maintain a concentration of 5.0×10⁵ vc/ml in the flask. When the volume of the shaker flask reached 50 ml and the cell density reached 1.0×10⁶ vc/ml, the culture was transferred to a 250 ml shake flask and diluted to 5.0×10⁵ vc/ml in 100 ml. When the cell density again reached 1.0×10⁶ vc/ml, the culture split and half transferred to another 250 ml shake flask and diluted to 2.0×10⁵ vc/ml. These steps of permitting the seed train to expand was continued until a sufficient volume was achieved to obtain a seeding density of 2.0×10⁵ vc/ml in the bioreactor. The ideal inoculum ratio (volume of inoculum culture/reactor liquid volume after inoculation) was judged to be about 10 to 20%. Accordingly, the cell density in the inoculum culture was adjudged to be ideally between 1.0×10⁶ vc/ml and 2.0×10⁶ vc/ml. The age of the inoculum culture was approximately 3 days old.

### Example 5

Medium for Use in the Production Bioreactor

A. Batch Medium

The batch medium for NIF1 production was prepared by combining

1.00 liter CHO-III-PFM with glucose (Life Technologies, Custom Formula 98-0289; with 3.45 g/l D-glucose; without hypoxanthine, thymidine, L-glutamine);

10.00 ml/l HT supplement (Life Technologies);

1.50 g/l yeast extract (Bacto, Difco/Becton-Dickinson); and

0.50 ml/l phenol red (0.5% (w/v) solution, Sigma).

B. Nutrient Feed 1

For use as nutrient feed 1, 200 grams glucose (from cerelose, Corn Products International) was dissolved in deionized water to make one liter of solution. This glucose feed is used to control the glucose concentration in the reactor at a concentration of approximately 1.5 to 2.5 g/l.

C. Nutrient Feed 2

For use as nutrient feed 2, the following components are combined:

1.0 liter CHO-III-PFM 5× (adjust to pH 7.4) (Life Technologies, Custom Formula 99-0180; 5× with 1× L-cystine, 3× L-tyrosine; without glucose, hypoxanthine, thymidine, L-glutamine, sodium bicarbonate, and sodium chloride).

50 ml/l HT supplement (Life Technologies, Catalog No. 11067-030; and 100×=10 mM sodium hypoxanthine, 1.6 mM thymidine).

7.50 grams yeast extract (Difco, Bacto/Becton-Dickinson).

The HT supplement and yeast extract was added to the CHO-III-PFM 5×. The components were mixed until completely dissolved, the pH was then adjusted to 7.4 using 5N sodium hydroxide, then the resulting solution was sterile filtered.

### Example 6

Operation of 2-Liter Stirred Tank Bioreactors

The production of NIF1 was performed in a 2-liter Wheaton bioreactor (B. Braun Biotech Inc., Allentown, Pa.), controlled via a Foxboro IA (Intelligence Application) computer system (Foxboro Company, Foxboro, Mass.). After sterilization of the bioreactor, one liter of sterile conditioning solution, glutamine-free DMEM (Life Technologies/GibcoBRL) was added. After four hours, the rinse medium was replaced with one liter of fresh, sterile production batch medium, as prepared in Example 5A. The temperature of the medium was allowed to stabilize at 36.5±1° C., and the pH was adjusted to pH 7.4. To obtain a target density in the reactor of approximately 2.0×10⁵ viable cells/ml, a volume of about 200 ml inoculum culture was added to obtain a 1.2 liter initial liquid volume.

A. Operational Setpoints

The following parameters for operation of the bioreactor were put in place.

Agitation: 100 rpm (4-inch diameter single plastic vertical blade).

pH: 7.40±0.15 (Control agents: CO₂ gas and a solution of 7.5% (w/v) NaHCO₃).

Dissolved oxygen concentration: 60% ±5% air saturation (Control agents: O₂ and N₂).

Temperature: 36.5° C.

Nutrient Feed 1: 200 g/l glucose solution—fed at about 0.0 to about 6.0 g/(liter-day).

Gas flow: 200-300 ml/min constant air flow to the headspace. CO₂ and O₂ (for safety purposes 60% O₂ in N₂ was employed) were sparged into the culture on demand to control pH and dissolved oxygen. N₂ was directed to the headspace at moments where the dissolved oxygen concentration exceeded its upper limit (65%).

Nutrient Feed 2 was fed continuously at a constant rate of 30 ml/day (i.e., at a rate of 25 ml per liter of culture at inoculation per day). This feed was started simultaneously with the glucose feed at 48 hours and maintained at this level over the course of the production run.

B. Sampling and Maintenance

The bioreactor was sampled immediately after inoculation. The following measurements were taken: the initial cell density and viability; the off-line pH; the initial glucose concentration; the initial lactate concentration; the initial ammonia concentration; and the initial osmolality. The on-line pH was adjusted when necessary. The bioreactor was covered in black plastic to protect the medium from light.

The bioreactor was sampled daily for the following parameters: cell density; culture viability; off-line pH; glucose concentration; lactate concentration; ammonia concentration; osmolality; and mature NIF1 concentration (starting at 4 days). The glucose concentration was maintained between 0.1 and 3.0 g/liter using Nutrient Feed 1. The feed with Nutrient Feed 1 began after 48 hours with an initial feed rate of approximately 2.0 g/(liter-day), or approximately 2.0 to 3.0 g glucose per 10⁹ viable cells per day) using a calibrated pump connected to an on/off timer using a 30 minute cycle. The glucose feed rate was adjusted each morning as necessary. The required feed rate usually remained within the range from 0.0 to 6.0 g/l-day. The constant Nutrient Feed 2 was started simultaneously with the glucose feed.

C. NIF1 Production Profile Using PFG01

The following measurements set forth in Table IV below were observed for the run of the production of NIF1 using PFG01 cells.

TABLE IV

| | | Cell | | | | | | |
|---|---|---|---|---|---|---|---|---|
| | | Count | Viability | NIF1 | Glucose | Lactate | Ammonia | Osmolality |
| Day | pH | (vc/ml) | (%) | (Units/ml) | (g/l) | (mmol/l) | (mmol/l) | (mOsm/kg) |
| 0 | 7.55 | 2.75E+05 | 100.0 | | 2.74 | | | |
| 1 | 7.53 | 1.60E+05 | 94.1 | | 2.76 | 6 | 0.584 | 315 |
| 2 | 7.43 | 4.25E+05 | 98.6 | | 1.89 | | | |
| 3 | 7.38 | 1.11E+06 | 100.0 | | 0.24 | 33 | 0.828 | 326 |
| 4 | 7.26 | 2.17E+06 | 96.6 | 0.5 | 1.30 | 54 | 0.626 | 350 |
| 5 | 7.35 | 2.66E+06 | 95.0 | 0.9 | 1.36 | 65 | 0.410 | 378 |
| 6 | 7.31 | 3.36E+06 | 96.0 | 1.6 | 1.98 | 70 | 0.536 | 391 |
| 7 | 7.32 | 3.12E+06 | 93.9 | 2.1 | 2.64 | 77 | 0.566 | 403 |
| 8 | 7.39 | 2.50E+06 | 86.8 | 2.7 | 2.34 | 78 | 0.414 | 405 |
| 9 | 7.24 | 1.50E+06 | 94.9 | 3.3 | 2.40 | | | |
| 10 | 7.38 | 1.50E+06 | 86.9 | 3.9 | 2.38 | | | |
| 11 | 7.39 | 1.88E+06 | 76.4 | 4.5 | 2.43 | 80 | 0.458 | 421 |
| 12 | 7.35 | 2.20E+06 | 68.8 | 5.0 | 2.86 | 81 | 0.652 | 426 |

The cell count and viability measurements were measured in accordance with the Trypan Blue Dye Exclusion method as set forth in Cell and Tissue Culture: Laboratory Procedures in Biotechnology, A. Doyle and J. B. Griffiths, eds. (John Wiley & Sons, Ltd., 1998). The NIF1 titer was measured by the protocol set forth above in the Detailed Description of the Invention. The glucose, lactate and ammonia measurements were conducted using a Kodak Biolyzer Rapid Analysis System. The osmolality was measured using an Advanced Micro Osmometer, (Model 3330, Advanced Instruments, Inc., Norwood, Mass.).

### Example 7

NIF1 Sialylation/Glycosylation Profile

The NIF1 harvested from a number of different bioreactor runs were tested for degree of sialylation/glycosylation, and compared with the results for a standard sample. The standard NIF1 (STD) was obtained from the cells of Example 1 that were adapted to suspension growth as set forth in Example 2, but nourished on media containing bovine serum albumin. Sialylation/glycosylation profiles were examined via the methods set forth in Webster et al., Xenobiotica, 29(11), pp. 1141-55 (1999) as follows.

A. Desialylation of NIF1

The procedure for desialylation of NIF1 uses acid hydrolysis to release the sialic acid. NIF1 samples (2 mg/ml) were desialylated by the addition of 0.2N HCl (1:1 V/V) and heated at 80° C. for 1 hour. The sialic acid liberated by the acid hydrolysis reactions is determined using the thiobarbituric acid method developed by Warren, J. Biol. Chem., 234, pp. 1971-5 (1959). The incubation was terminated when no further increase in free sialic acid was observed.

B. Total Sialic Acid Determination

The sialic acid residues were released from NIF1 using acid hydrolysis (part A immediately above) and the predominant sialic acid associated with the glycans of the NIF protein, 5-acetylneuramic acid (neu5ac), was analyzed by ion chromatography with pulsed amperometric detection. Purified NIF1 samples were diluted to a concentration of 0.1 mg/ml. The sample (200 μl) was then mixed with 200 μl of 0.2N HCl and heated at 80° C. for 1 hour. The sample was then cooled in an ice bath for 10 minutes and centrifuged. The supernatant (20 μl), containing 5-acetylneuramic acid, was analyzed using a Dionex Carbopac (PA-10 4.6×250 mm column (guard column 4.6×50 mm Carbopac (PA-10) with a 0.2M NaOH and 0.05M C₂H₃O₂Na mobile phase (flow rate=0.7 ml/min, mobile phase conditioned with a Dionex Ion Pac ATC-1 mobile phase conditioner) and detection was carried out using a Dionex ED40 detector using the optimized carbohydrate waveform setting. The 5-acetylneuramic acid standard has a retention time of 10 minutes under the above HPLC conditions. A wash step was performed after the elution of 5-acetylneuramic acid (0.2 M NaOH and 0.3 M C₂H₃O₂Na for 5 minutes) and the column was re-equilibrated for 30 minutes before the next injection. The data are presented as a percentage of a control batch (i.e., standard sample of NIF1 (STD) prepared from Example 1 cells adapted to suspension but made in the presence of bovine serum albumin) and an increase in the value presented for sialylation represent an increase in the amount of sialic acid on the NIF1 molecule. The total sialylation data is presented in Table V.

C. Oligosaccharide Charge Profile Assay

N-linked oligosaccharides are released from NIF1 using the enzyme peptide-N-Glycosidase F (PNGase-F). The released oligosaccharides are labeled with 2-aminobenzamide (2-AB) and separated on anion exchange chromatography. Purified NIF1 was first diluted to a concentration of about 0.05 mg/ml; 50 μl of the diluted NIF was denatured by the addition of 4 μl of 5% (w/v) SDS and 6 μl of 1.44M β-meracaptoethanol. After setting the sample aside for about 5 minutes, 20 μl of 7.5% NP-40 and 30 μl of (100 mM Na₂HPO₄, 10 mM EDTA-disodium, pH 7.6) buffer were added to the sample. Following this, 10 μl of 1 mU/ml PNGase-F was added and the sample was stored in an incubator for 18-24 hours at 37° C. Released oligosaccharides were separated from deglycosylated protein by ethanol precipitation, the supernatant was removed and dried. This dried sample containing oligosacharides were labeled with 2-AB using a labeling kit (5 μl of labeling reagent used to re-suspend sample, Signal 2-AB labeling kit (Oxford Glycoscience, product number K-404). At the end of the incubation excess labeling reagent was removed by chromatography on a hydrophilic membrane (supplied with the kit). The reagent mixture was loaded onto the disk in acetonitrile and excess reagent removed with sequential acetonitrile and acetonitrile/water washes. 2-AB labeled oligosaccharides were eluted from the disk with water and dried. The oligosaccharides labeled with 2-AB were then analyzed by anion exchange HPLC on a Glycosep C HPLC column (100×4.6 mm, Oxford Glycosystems). Oligosaccharides were eluted in a gradient from 80% water/20% acetonitrile to 80% 250 mM ammonium acetate pH 4.5/20% acetonitrile over 35 minutes, flow rate was 0.3 ml/min. Fluorescence detection was performed with an excitation wavelength of 330 nm and an emission wavelength of 420 nm. A typical chromatogram consists of peak clusters with uncharged species eluting first followed by mono-, di-, tri- and tetra-sialylated cluster.

A NIF1 batch (STD) was designated for profiling control. The sialylation profile data is presented in Table V.

### Example 8

NIF1 Pharmacokinetic Profile

The NIFs harvested from a number of different bioreactor runs were tested for pharmacokinetic clearance and half-life data, and compared with the results for a standard sample (STD) of NIF1 obtained from the cells of Example 1 that were adapted to suspension growth as set forth in Example 2, but made in the presence of animal protein (BSA). The results are listed in the following table as compared with a standard sample of NIF1.

A. Preparation of Animals

Jugular vein catheterized Fischer 344 rats were prepared by inserting a cannula (0.58 mm I.D., 0.9 mm O.D., polythene tubing, Portex Ltd.) into the jugular vein and the cannula was exteriorized at the back of the neck using classical veterinary techniques. During the surgery rats were anaesthetized with 70 mg/kg ketamine HCl (Vetalar, Parke-Davis Veterinary; 100 mg/ml)/10 mg/kg xylazine (Rompun injection 2%, Bayer) administered as an intra peritoneal injection. Following surgery, reversal of the xylazine was carried out using.a 1 ml/kg injection of 1 in 5 diluted Antisedan (Pfizer Animal Health, atipamezole; 5 mg/ml) administered as a subcutaneous injection. Analgesia was provided for the duration of the experiment (Buprenorphine HCl, 0.1 ml of a 1 in 4 dilution bf Vetergesic (Reckitt and Colman; 0.3 mg/ml)) administered subcutaneously. Following a recovery period of two days, rats were dosed into the tail vein (bolus) with NIF1 at a dose level of 2 mg/kg (doses were made up at a concentration of 2 mg/ml and administered on a 1 ml/kg basis). The use of jugular vein catheterized rats allowed for serial sampling and two rats were used, to determine the pharmacokinetic of each batch of NIF1. Blood samples (50 μl) were removed using the indwelling cannula into heparinized tubes at the following time points: Pre Dose, 0.25, 1, 2, 4, 8, 12, 24 and 48 hours. The blood samples were centrifuged, the plasma removed and stored frozen for subsequent analysis. Plasma samples were analyzed for NIF1 using a Delfia immunoassay (Example 8C).

B. Isolated Perfused Rat Liver Preparations

The isolated perfused rat liver (IPRL) preparation was carried out using the methodology detailed in Gardner et al., Xenobiotica, 25, pp. 185-187 (1995). Male rats selected at a weight of approximately 250 g were anaesthetized (Intraval) and surgery performed to cannulate the bile duct, hepatic portal vein and superior vena cava.

A perfusate consisting of a pH 7.4 Krebs high bicarbonate buffer (61%) containing washed human red cell (13%) and 10% (w/v) bovine serum albumin (26%) was perfused through the liver at a flow rate of 15 ml/min. The perfusate was oxygenated using 95% oxygen/5% carbon dioxide and enters the liver via the hepatic portal vein, exiting via the vena cava. The IPRL was run in recirculating mode using a total perfusate volume of 150 ml. A solution containing taurocholic acid (24 mg/ml) was infused at a rate of 1.33 ml/h for the duration of the experiment to maintain bile flow. Asialo NIF1 (0.25 mg, Example 7A) was administered to the reservoir and perfusate samples were withdrawn from the reservoir after 2, 5, 10, 15, 20, 30, 45, 60, 75 and 90 minutes. Bile was collected for the duration of the experiment (0-90 minutes). For the competition studies asialo NIF (0.25 mg) was co-administered with asialo fetuin (10 mg, Sigma A1908). The perfusate samples were centrifuged and the supernatant removed and stored frozen for subsequent analysis. Bile samples were also stored frozen for analysis. The NIF1 concentration in IPRL perfusate and bile samples was determined using the Delfia immunoassay detailed for plasma (Example 8C).

C. Analysis of Plasma Samples for NIF1

Plasma samples were analyzed using a Dissociation Enhanced Lanthanide Fluorescence Immunoassay (Delfia). The assay is a “sandwich non-competitive immunoassay” using europium-labeled monoclonal anti-NIF antibody as detection reagent. Polyclonal rabbit anti-NIF antibodies are bound to plates coated with anti-rabbit antibody. NIF in samples or standards then binds to the polyclonal antibodies and finally europium-labeled monoclonal antibody binds to another epitope on the bound NIF. Europium is determined after addition of “enhancement” solution. In order to perform this assay 5 μl of plasma was required.

The assay range in plasma was 0.1-40 μg/ml. The accuracy of this assay was evaluated and the cumulative variations were 10.5, 3.4, 6.3% at 0.1, 3 and 40 μg/ml, respectively. The day to day performance of the assay was monitored using quality control samples. Delfia immunoassays are well characterized and have been previously used to determine the plasma concentrations of many protein containing molecules including; interferons (Ronnblom et al., APMIS, 105, pp. 531-536 (1997)), apolipoprotein D (Knipping et al., J. Immunological Methods, 202, pp. 85-95 (1997)), thyroglobulin (Dai et al., Clinical Biochemistry, 29, pp. 461-465 (1996) and lipoprotein lipase (Wicher et al., J. Immunological Methods, 192, pp. 1-11 (1996)).

D. Pharmacokinetic Analysis

The pharmacokinetics were determined using standard algorithms. The elimination rate constant (Kₑ₁) was determined from the plot of concentrations in plasma verses time using linear regression of the log (plasma concentration) versus time. The half-life determined using the following equation: Half-life=(Ln2)Kₑₗ-area under the plasma concentration time curve (AUC) was calculated from time zero to the last data point using the linear trapezoidal rule. The AUC was extrapolated to infinity using the elimination rate constant. Clearance was calculated using the relationship: Dose divided by AUC_{(0-∞)}. Volume of distribution was calculated by the relationship: Clearance divided by the elimination rate constant.

The hepatic extraction value in the IPRL was calculated by dividing the clearance value obtained in the IPRL by the IPRL flow rate (15 ml/min) and multiplying this value by 100. The results of the clearance and half-life determination are set forth in Table V below. The NIF1 titer was measured by the method set forth above in the description.

TABLE V

| | | Titer | Sielylation Profiles | | | | | | | Clearance | Half- |
|---|---|---|---|---|---|---|---|---|---|---|---|
| | | Units | Total | | | | | | Tri + | (ml/min/ | Life |
| Run | Day | /ml) | (%) | Zero | Mono | Di | Tri | Tetra | Tetra | kg) | (h) |
| STD | | 1.0 | 100 | 5.8 | 14.3 | 21.1 | 26.3 | 32.5 | 58.8 | 0.08 | 11.5 |
| A | 10 | 3.8 | 85.6 | 5.3 | 18.5 | 26.9 | 29.1 | 20.3 | 49.4 | 0.08 | 9.7 |
| B | 12 | 4.9 | 81.2 | 7.9 | 20.2 | 27.0 | 27.5 | 17.4 | 44.9 | 0.08 | 9.4 |
| C | 10 | 3.4 | 85.7 | 6.4 | 17.4 | 25.8 | 29.3 | 21.1 | 50.4 | 0.07 | 9.5 |
| D | 10 | 4.2 | 81.3 | 8.5 | 19.5 | 25.3 | 28.8 | 17.9 | 46.7 | 0.09 | 9.2 |
| E | 11 | 4.0 | 85.4 | 7.2 | 18.5 | 24.5 | 28.8 | 21.0 | 49.8 | 0.12 | 11.9 |
| F | 11 | 4.0 | 94.3 | 3.2 | 14.8 | 24.5 | 32.7 | 24.8 | 57.5 | 0.09 | 14.3 |

| | | | | | | | | | | |
|---|---|---|---|---|---|---|---|---|---|---|
| Average | 4.0 | 85.6 | 6.4 | 18.2 | 25.7 | 29.4 | 20.4 | 49.8 | 0.09 | 10.7 |
| (Runs A- | | | | | | | | | | |
| F) | | | | | | | | | | |

1. A process for the preparation of Neutrophil Inhibitory Factor comprising the step of growing a cell line expressing Neutrophil Inhibitory Factor in an animal component-free medium selected from the group consisting of an inoculum growth medium, a production growth medium and a nutrient feed to give a production culture.

2. A process according to claim 1 wherein the Neutrophil Inhibitory Factor is the protein of SEQ. ID. NO. 3.

3. A process for the according to claim 2 wherein the protein is glycosylated and has a relative molecular weight of about 38.3 to about 64.1 kDa.

4. A process according to claim 2 wherein the protein is about 5 to about 25% mono-sialylated; about 10 to about 30% di-sialylated, about 15 to about 35% tri-sialylated, about 15 to about 45% tetra-sialylated and about 1 to about 20% non-sialylated.

5. A process according to claim 1 wherein the animal component-free production growth medium comprises:
(i) a CHO-III-PFM/glucose solution;
(ii) a sodium hypoxanthine/thymidine solution; and
(iii) yeast extract.

6. A process according to claim 1 wherein the animal component-free production growth medium comprises:
(i) CHO-III-PFM/glucose solution;
(ii) about 5 to about 20 ml per liter (i) of a 10 mM sodium hypoxanthine/1.6 mM thymidine solution; and
(iii) about 0.5 to about 5.0 grams per liter (i) yeast extract.

7. A process according to claim 1 wherein the animal component-free production growth medium comprises:
(i) CHO-III-PFM/glucose;
(ii) 10.0 ml per liter (i) of a 10 mM sodium hypoxanthine/1.6 mM thymidine solution; and
(iii) 1.5 grams per liter (i) yeast extract.

8. A process according to claim 1 further comprising the steps of:
(a) providing an inoculum prepared by incubating a cell line expressing Neutrophil Inhibitory Factor in an animal component-free inoculum growth medium; and
(b) transferring said inoculum to a vessel containing an animal component-free production growth medium.

9. A process according to claim 8 wherein the inoculum growth medium comprises:
(i) a CHO-III-PFM/glucose;
(ii) a sodium hypoxanthine/thymidine solution;
(iii) an amino acid solution comprising acids selected from the group consisting of L-aspartic acid, L-glutamic acid, L-asparagine, L-proline, L-serine, and L-methionine;
(iv) optionally an L-methionine sulphoximine solution; and
(v) an L-cysteine solution.

10. A process according to claim 8 wherein the inoculum growth medium comprises:
(i) CHO-III-PFM/glucose solution;
(ii) about 5 to about 20 ml per liter (i) of a 10 mM sodium hypoxanthine/1.6 mM thymidine solution;
(iii) about 5 to about 30 ml per liter (i) of an amino acid solution comprising L-aspartic acid (3.0 g/l), L-glutamic acid (2.5 g/l), L-asparagine (10.0 g/l), L-proline (1.25 g/l), L-serine (3.0 g/l), and L-methionine (1.5 g/l);
(iv) about 0 to about 75 μmol per liter (i) of L-methionine sulphoximine; and
(v) about 10 to about 40 mg per liter (i) of L-cysteine.

11. A process according to claim 8 wherein the inoculum growth medium comprises:
(i) CHO-III-PFM/glucose;
(ii) 10.0 ml per liter (i) of a 10 mM sodium hypoxanthine/1.6 mM thymidine solution;
(iii) 20.0 ml per liter (i) of an amino acid solution comprising L-aspartic acid (3.0 g/l), L-glutamic acid 2.5 g/l), L-asparagine (10.0 g/l), L-proline (1.25 g/l), L-serine (3.0 g/l), and L-methionine (1.5 g/l);
(iv) optionally 1.0 ml per liter (i) of a 25 mM L-methionine sulphoximine solution; and
(v) 25.0 mg per liter (i) of L-cysteine.

12. A process according to claim 8 further comprising the step:
(c) feeding the production culture with at least one nutrient feed.

13. A process according to claim 12 wherein step (c) includes a first nutrient feed and a second nutrient feed.

14. A process according to claim 13 wherein the first nutrient feed is a nutrient feed comprising an aqueous solution of about 100 to about 500 grams of glucose per liter.

15. A process according to claim 13 wherein the first nutrient feed is a nutrient feed comprising an aqueous solution of about 200 grams of glucose per liter.

16. A process according to claim 15 wherein the first nutrient feed is added at a rate of about 0.0 to about 6.0 grams of glucose per liter growth medium per day.

17. A process according to claim 13 wherein the second nutrient feed comprises
(i) a CHO-III-PFM (5×) solution with 1× L-cystine, 3× L-tyrosine and without glucose, hypoxanthine, thymidine, L-glutamine, sodium bicarbonate, sodium chloride;
(ii) 25 to 100 ml per liter of solution (i) of a 10 mM sodium hypoxanthine/1.6 mM thymidine solution; and
(iii) 5 to 20 grams per liter of solution (i) yeast extract.

18. A process according to claim 13 wherein the second nutrient feed comprises
(i) CHO-III-PFM (5×) solution with 1× L-cystine, 3× L-tyrosine and without glucose, hypoxanthine, thymidine, L-glutamine, sodium bicarbonate, sodium chloride;
(ii) 50 ml per liter of solution (i) of a 10 mM sodium hypoxanthine/1.6 mM thymidine solution; and
(iii) 7.5 grams per liter of solution (i) yeast extract.

19. A process according to claim 18 wherein the second nutrient feed is fed to the reactor continuously at a rate of approximately 25 ml/liter-day.

20. A process according to claim 13 wherein said first nutrient feed comprises about 100 to about 500 grams per liter glucose and said second nutrient feed comprises (1) a CHO-III-PFM (5×) solution, (2) about 25 to about 100 ml per liter (1) of a 10 mM sodium hypoxanthine/1.6 mM thyandine solution; and (3) about 5 to about 5 grams per liter (1) yeast extract.

21. A process according to claim 12 wherein the nutrient feed is a nutrient feed comprising an aqueous solution of about 100 to about 500 grams of glucose per liter.

22. A process according to claim 12 wherein the nutrient feed is a nutrient feed comprising an aqueous solution of about 200 grams of glucose per liter.

23. A process according to claim 22 wherein the nutrient feed is added at a rate of about 0.0 to about 6.0 grams of glucose per liter growth medium per day.

24. A neutrophil inhibitory factor made by the process of claim 1.

25. An animal component-free production growth medium comprising:
(i) a CHO-III-PFM/glucose solution;
(ii) a sodium hypoxanthine/thymidine solution; and
(iii) yeast extract.

26. A medium according to claim 25 comprising:
(i) CHO-III-PFM/glucose solution;
(ii) about 5 to about 20 ml per liter (i) of a 10 mM sodium hypoxanthine/1.6 mM thymidine solution; and
(iii) about 0.5 to about 5.0 grams per liter (i) yeast extract.

27. A medium according to claim 25 comprising:
(i) CHO-III-PFM/glucose solution;
(ii) 10.0 ml per liter (i) of a 10 mM sodium hypoxanthine/1.6 mM thymidine solution; and
(iii) 1.5 grams per liter (i) yeast extract.

28. A method for the preparation of recombinant proteins comprising the cultivation of mammalian cells expressing an exogenous recombinant protein in the animal component-free growth medium of claim 25.

29. A method according to claim 28 wherein the mammalian cells are Chinese Hamster Ovary cells transfected with a glutamine synthetase plasmid vector containing the DNA coding region for the recombinant protein.

30. A method according to claim 29 wherein the vector is a glutamine synthetase/methionine sulfoximine co-amplification vector selected from pEE14 and pEE14.1.

31. An inoculum growth medium comprising:
(i) a CHO-III-PFM/glucose solution;
(ii) a sodium hypoxanthine/thymidine solution;
(iii) an amino acid solution comprising acids selected from the group consisting of L-aspartic acid, L-glutamic acid, L-asparagine, L-proline, L-serine, and L-methionine;
(iv) optionally an L-methionine sulphoximine solution; and
(v) an L-cysteine solution.

32. An inoculum growth medium according to claim 31 comprising:
(i) CHO-III-PFM/glucose solution;
(ii) about 5 to about 20 ml per liter (i) of a 10 mM sodium hypoxanthine/1.6 mM thymidine solution;
(iii) about 5 to about 30 ml per liter (i) of an amino acid solution comprising L-aspartic acid (about 3.0 g/l), L-glutamic acid (about 2.5 g/l), L-asparagine (about 10.0 g/l), L-proline (about 1.25 g/l), L-serine (about 3.0 g/l), and L-methionine (about 1.5 g/l);
(iv) about 0 to about 75 μmol per liter (i) of an L-methionine sulphoximine; and
(v) about 10 to about 40 mg per liter (i) of L-cysteine.

33. An inoculum growth medium according to claim 32 comprising:
(i) CHO-III-PFM/glucose solution;
(ii) 10.0 ml per liter (i) of a 10 mM sodium hypoxanthine/1.6 mM thymidine solution;
(iii) 20.0 ml per liter (i) of an amino acid solution comprising L-aspartic acid (3.0 g/l), L-glutamic acid 2.5 g/l), L-asparagine (10.0 g/l), L-proline (1.25 g/l), L-serine (3.0 g/l), and L-methionine (1.5 g/l);
(iv) optionally 1.0 ml per liter (i) of a 25 mM L-methionine sulphoximine solution; and
(v) 25.0 mg per liter (i) of L-cysteine.

34. A nutrient feed comprising
(i) a CHO-III-PFM (5×) solution with 1 × L-cystine, 3x L-tyrosine and without glucose, hypoxanthine, thymidine, L-glutamine, sodium bicarbonate, sodium chloride;
(ii) 25 to 111 ml per liter of solution (i) of a 10 mM sodium hypoxanthine/1.6 mM thymidine solution; and
(iii) 5 to 20 grams per liter of solution (i) yeast extract.

35. The cell line PFG-01 (ATCC PTA-2503).

36. A method for the preparation of Neutrophil Inhibitory Factor comprising culturing the cell line of claim 35 under conditions promoting expression of Neutrophil Inhibitory Factor and recovering the Neutrophil Inhibitory Factor.

37. A method according to claim 36 wherein the Neutrophil Inhibitory factor comprises the amino acid sequence of SEQ. ID. NO. 3.

38. A Neutrophil Inhibitory Factor prepared by culturing the cell line PFGOl (ATCC PTA-2503).

39. A Neutrophil Inhibitory Factor prepared by the method of claim 36 or 37.

40. A process according to any of claims 1 to 23 wherein said cell line is PFG01 (ATCC PTA-2503).

41. A Neutrophil Inhibitory Factor made by the process of any of claims 2 to 23.

42. An isolated Neutrophil Inhibitory Factor having neutrophil inhibitory activity and comprising the amino acid sequence of SEQ. ID. NO. 3 that is produced by cell line PFG01 (ATCC PTA-2503).
